(19) United States
(12) Patent Application Publication
(10) Pub. No.: US 2008/0152630 A1
(43) Pub. Date: Jun. 26, 2008

(54) Method of generation and expansion of tissue-progenitor cells and mature tissue cells from intact bone marrow or intact umbilical cord tissue

(76) Inventors: Irene Ginis, Beit Shemesh (IL); Aharon Schwartz, Mavasert Zion (IL); Doron Shinar, Kfar Sava (IL); Mitchell Shirvan, Herzylia (IL)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/001,086

(22) Filed: Dec. 7, 2007

Related U.S. Application Data
(60) Provisional application No. 60/868,969, filed on Dec. 7, 2006.
(60) Provisional application No. 60/972,309, filed on Sep. 14, 2007.

(51) Int. Cl. A61K 35/00; C12N 5/06; A61P 43/00; C12Q 1/68

(52) U.S. Cl. 424/93.7; 435/377; 435/6

(57) ABSTRACT

Disclosed are compositions and methods of generating and expanding tissue-progenitor cells or mature tissue cells in culture, comprising culturing intact bone marrow or intact umbilical cord tissue in a cell differentiation medium whereby tissue-progenitor cells or mature tissue cells are generated from mesenchymal stem cells and various progenitor cells present in the intact bone marrow or intact umbilical cord tissue and expanded, and methods of using the tissue-progenitor cells or mature tissue cells in processes of tissue repair or regeneration.

### CROSS REFERENCE

This application claims the benefit of the filing dates of U.S. Provisional Patent Application Nos. 60/868,969 filed Dec. 7, 2006 and 60/972,309 filed Sep. 14, 2007, and the disclosure of which is hereby incorporated herein by reference.

### TECHNICAL FIELD

The present invention relates to generation and expansion of tissue-progenitor cells or mature tissue cells in vitro, and methods of repairing or regenerating tissue using the cells.

### BACKGROUND OF THE INVENTION

Bone tissue repair accounts for approximately 500,000 surgical procedures per year in the United States alone (Geiger et al., 2003). Similarly, injuries and degenerative changes in the articular cartilage are, in essence, a significant cause of morbidity and diminished quality of life, with arthritis ranking second only to cardiovascular disease (Walker J M, 1998) where improvement of neovascularization is an important therapeutic option (Kawamoto A, et al., 2001). Osteogenesis, chondrogenesis, angiogenesis, and chronic wound healing are all natural repair mechanisms that occur in the human body. However, there are critical sizes of defects greater than which these tissues will not regenerate (e.g., after significant osteotomy because of bone cancer). In addition, about 10% of all bone fractures result in nonunion because of various systemic conditions. In these cases there is a need for therapeutic intervention. For example, for bone and cartilage repair the defect is usually filled with permanent or biodegradable porous scaffold unless a bone/cartilage transplant is used (Giannoudis et al., 2005). In recent years, it has been recognized that the scaffolds can be seeded with specific stem cells that will increase the rate of regeneration (Caplan, 2005). Similarly, stem cells and their partially differentiated progenitors have been injected in the site of myocardial ischemia (Schueller P O et al., 2006) or brain ischemia (Chen J and Chopp M, 2006), resulting in clinical improvement.

Although experiments aimed to produce various tissues from embryonic stem cells are in progress, adult mesenchymal stem cells (adult MSCs or AMSCs) found in the bone marrow (Vaananen H K. 2005), peripheral blood (Huss R et al., 2000), adipose tissue (Zuk P A et al., 2001), muscle, connective tissue and dermis (Young H E et al., 2001; Asahara A et al., 2001) are currently considered a feasible source of autologous or allogeneic stem cells for tissue engineering. Cells with features of MSCs have also been isolated from umbilical cord blood (Erices A, et al., 2000; Bieback K et. al., 2004; Kern S et al., 2006) and umbilical cord matrix (Mitchell K E et al., 2003).

A commonly accepted hypothesis is that adult stem cells emerge during development and then are somehow “conserved” in the adult organism for tissue/organ maintenance and repair (Ratajczak et al., 2004; da Silva Meirelles et al., 2006). If such conservation occurs at various stages of development, then the resulting stem cells should differ in degree of maturity and differentiation potential. Bone marrow is an important source of AMSCs, which are capable of differentiation into tissues such as bone, fat, cartilage and connective tissue that arise from mesenchymal origin during development.

In addition to differentiation pathways common to mesenchymal lineages, recent in vivo and in vitro studies have highlighted the potential of MSCs from bone marrow (Deng J, et al., 2006), umbilical cord matrix (Mitchell K E et al., 2003), and cord blood (Habich A et al., 2006; El-Badri N S, et al., 2006) to develop into cells that express neuronal markers.

Variability of AMSCs in the bone marrow is demonstrated by the finding that various methods of isolation and culture of bone-marrow derived AMSCs yield stem cells with different phenotypes and differentiation potentials. These cells even receive different names, such as bone marrow stromal stem cells (BMSSC) (Bianco and Robey, 2000), recycling stem cells (RS-1 and RS-2) (Colter et al., 2001), marrow isolated adult multilineage inducible (MIAMI) cells (D'lppolito et al., 2004), multipotent adult progenitor cells (MAPC) (Verfaillie, 2005), and tissue committed stem cells (TCSC) (Ratajczak et al, 2004; Bedada et al., 2006). These cell populations can represent different points of a hierarchy or a continuum of differentiation.

There is no common isolation method that is capable of yielding a connected series of stem cells and/or their progenitors capable of differentiating into a specific tissue. Each existing method results in unwanted cell populations while losing part of the yield of desirable cells. The most common method of isolation of AMSCs from bone marrow or umbilical cord blood, referred to as adhesion selection, relies on the ability of AMSCs to adhere to plastic surfaces. However, this condition favors the expansion of not only stem cells but of other types of non-hematopoietic cells, e.g., stromal fibroblasts. This results in a heterogeneous population of cells including some cell types that are not desired. For example, although osteogenic differentiation is believed to be the default lineage of AMSCs, only 60% of colonies obtained through adhesion selection form bone after transplantation in vivo (Kuznetsov et al., 1997).

Another method of isolation of AMSCs is based on negative immunoselection and elimination of hematopoietic cells. The resulting population of non-hematopoietic cells is also heterogeneous (Tondreau et al., 2004). To date, there is no consensus regarding specific markers of AMSCs.

Nevertheless, attempts have been made to perform immunosorting of AMSCs based on one or two markers commonly found on AMSCs such as CD105 (Aslan et al., 2006), STRO-1 (Encina et al., 1999), or LNGFR (Quirici et al., 2002). In the absence of specific markers, MSCs are characterized by simultaneous expression of certain known markers (CD105, CD44, CD166, CD73, CD90 and others). Thus the method of immunosorting based on one marker also results in heterogeneous population with lower yield of actual stem cells.

Transplantation of MSC and more differentiated progenitors for tissue repair and in particular for repair of large bone defects and of non-union bone fractures often require a carrier or scaffold. In addition to the quality of the transplanted cells and their purity and degree of differentiation, the characteristics of the carrier or scaffold are of great importance. For example, survival of bone progenitors (BP) transplanted on a scaffold could be affected by insufficient graft vascularization. Ingrowth of blood vessels from the edges of broken bone or from surrounding soft tissues might be too slow to support survival of cells seeded deep into a scaffold.

### SUMMARY OF THE INVENTION

A first aspect of the present invention is directed to a method of generating and expanding more or less differentiated tissue-progenitor cells or mature tissue cells in culture, comprising culturing intact bone marrow or intact umbilical cord tissue in a cell differentiation medium whereby tissue-progenitor cells or mature tissue cells are generated from all cellular sources, such as mesenchymal stem cells (MSCs) and various progenitor cells, present in the intact bone marrow or intact umbilical cord tissue that are capable of differentiation, and expanded. Given the extensive multipotency of MSCs, the methods of the present invention may be used to generate and expand cells that can be used for the repair or regeneration of a variety of tissues, including bone, cartilage, heart, vasculature (e.g., smooth muscle)/endothelium, nerve tissue, pancreatic tissue, skin and adipose tissue.

A second aspect of the present invention is directed to a method of tissue repair or regeneration, comprising:

(a) culturing intact bone marrow or intact umbilical cord tissue in a cell differentiation medium whereby tissue-progenitor cells or mature tissue cells are generated and expanded;

(b) harvesting the tissue-progenitor cells or mature tissue cells; and

(c) transplanting the tissue-progenitor cells or mature tissue cells in a patient in need thereof.

A third aspect of the present invention is directed to a composition, comprising intact bone marrow or intact umbilical cord tissue and a cell differentiation medium, which upon culturing achieves generation and expansion of tissue-progenitor cells or mature tissue cells from mesenchymal stem cells and/or various progenitor cells present in the intact bone marrow or intact umbilical cord tissue.

Broadly, the present invention provides a much simplified and more efficient method of generating or differentiating and expanding progenitor cells of various tissues in vitro. More specifically, using intact bone marrow or intact umbilical cord tissue eliminates the need for costly and detailed physical and/or chemical pretreatment of the bone marrow or umbilical cord tissue in order to isolate or extract stem cells, such as MSCs, thus eliminating the need for reagents required for isolation of stem cells (thus reducing costs, and the time need to satisfy FDA requirements and regulations) and makes quality control (QC) easier because tissue-progenitor cells express better-defined markers as compared to undifferentiated AMSCs. The method also produces nearly homogeneous populations of expanded cells. Even further, the method saves production time and improves the yield and viability of the generated and expanded cell types by reducing cell injury and loss caused by isolation procedures and by allowing the differentiation process to occur in the native environment of the cell.

### BRIEF DESCRIPTION OF THE DRAWINGS

Other advantages of the present invention are readily appreciated as the same becomes better understood by reference to the following detailed description when considered in connection with the accompanying drawings, wherein:

FIG. 1 depicts the proliferation rate of BP generated in three ways: by incubation of unprocessed bone marrow either with growth medium for 2 weeks (legend—GM) or with differentiating medium for two weeks (legend—DM), or by incubation with growth medium for one week and then with differentiating medium for another week (legend GM-DM). Undifferentiated MSC cultures produced from Ficoll-isolated mononuclear cells (MNC) using conventional method were used for comparison. All the cells were trypsinized, replated into 24 wells and allowed to proliferate and differentiate in osteogenic differentiation medium for various times. (The results of measuring cell proliferation two experiments are shown in FIGS. 1A and 1B, respectively.)

FIG. 2 depicts alkaline phosphatase (ALP) activity in the same groups of cells, as described in FIG. 1.

FIG. 3 depicts calcium depositions in cultures of the same groups of cells, as described in FIG. 1.

FIG. 4 is a photograph illustrating the alizarin red staining of calcium deposits in the same groups of cells, as described in FIG. 1.

FIG. 5 presents the statistical data (A) and actual histograms (B-D) of flow cytometry analysis of bone-specific ALP activity in BP generated from unprocessed bone marrow and in conventional MSC undergoing differentiation.

FIG. 6 depicts (A) microphotographs of neuronal progenitors derived from unprocessed bone marrow (BM), (B) flow cytometry analysis of early neuronal markers: nestin and PSA-NCAM, and (C) Class III b-tubulin expression in bone marrow-derived neuronal progenitors.

FIG. 7 depicts the comparison of cell yields of BP derived from unprocessed BM either in 10% FCS or without serum in cell culture plates.

FIG. 8 depicts the comparison of cell yields of BP derived from unprocessed BM either in 10% FCS or without serum grown on various scaffolds.

FIG. 9 depicts the results of the quantitative assay of ALP activity in BP derived from unprocessed BM either in 10% FCS or without serum.

FIG. 10 depicts microphotographs of BP derived from unprocessed BM either in (A) 10% FCS or (B) without serum, and stained for ALP activity.

FIG. 11 depicts statistical data of flow cytometry analysis of ALP expression in BP derived from unprocessed BM either in the presence of 10% FCS or without serum.

FIG. 12 depicts microphotographs of BP derived from unprocessed BM either in (A) 10% FCS or (B) without serum, and stained with alizarin-red for calcium deposits.

FIG. 13 depicts production of osteoclasts from unprocessed bone marrow. Purple cells are osteoclast progenitors positive for TRAP. Also, a multinucleated mature osteoclast is seen.

FIG. 14 depicts statistical data of flow cytometry analysis of ALP expression in BP derived from unprocessed BM either on fibronectin-coated or BM plasma-coated tissue culture plates.

FIG. 15 depicts the comparison of ALP activity in BP derived by culturing of unprocessed bone marrow on a scaffold coated with fibronectin or with BM plasma.

FIG. 16 depicts the results of transplantation of BP produced from human intact BM into nude mice in model of critical size femoral defect.

### DETAILED DESCRIPTION

To facilitate understanding of the invention, some of the terms used herein are defined as follows:

“Whole bone marrow” (WBM) or “intact bone marrow” refers to whole bone marrow from any source, e.g., surgical waste, commercial WBM aspirates, donor allogeneic and autologous bone marrow aspirates, which has not been pretreated to specifically isolate, extract or concentrate MSCs.

“Intact umbilical cord tissue” refers to whole solid tissue from an umbilical cord, which has not been pretreated to specifically isolate, extract or concentrate MSCs. Intact umbilical cord tissue includes Wharton's jelly and/or umbilical cord blood.

“Marrow derived bone progenitors” (MDBP) are bone marrow cells committed to development into mature bone cells.

“Tissue-progenitor cells” refers to cells that are committed to differentiation into certain specialized cells of various tissues. These cells are tissue-specific and will proliferate to form specific tissues under proper conditions.

“Progenitor cells” are cells produced during differentiation of a stem cell that have a potential for differentiation into one or more lineages. They are less differentiated than “tissue progenitor cells” but more restricted in differentiation pathways compared to MSC, that are called multipotent.

The term “bone marrow plasma” refers to the supernatant of a whole bone marrow sample after centrifugation.

The term “osteogenic differentiation medium” refers to any medium which provides the necessary elements to allow differentiation of MSCs/progenitor cells present in intact bone marrow or umbilical cord tissue, into bone-progenitor cells, and expansion of those cells in vitro.

The term “neurogenic differentiation medium” refers to any medium which provides the necessary elements to allow differentiation of MSCs/progenitor cells present in intact bone marrow or umbilical cord tissue, into neuronal-progenitor cells, or neurons, and expansion of those cells in vitro.

The term “endothelial differentiation medium” refers to any medium which provides the necessary elements to allow differentiation of MSCs/progenitor cells present in intact bone marrow or umbilical cord tissue, into vasculature/endothelial-progenitor cells, and expansion of those cells in vitro.

The term “adipogenic differentiation medium” refers to any medium which provides the necessary elements to allow differentiation of MSCs/progenitor cells present in intact bone marrow or umbilical cord tissue, into adipose-progenitor cells, or adipocytes, and expansion of those cells in vitro.

The term “cardiomyogenic differentiation medium” refers to any medium which provides the necessary elements to allow differentiation of MSCs/progenitor cells present in intact bone marrow or umbilical cord tissue, into heart muscle progenitor cells, or cardiomyocytes, and expansion of those cells in vitro.

The term “pancreogenic differentiation medium” refers to any medium which provides the necessary elements to allow differentiation of MSCs/progenitor cells present in intact bone marrow or umbilical cord tissue, into progenitors of pancreatic β-cell cells, and expansion of those cells in vitro.

The term “chondrogenic differentiation medium” refers to any medium which provides the necessary elements to allow differentiation of MSCs/progenitor cells present in intact bone marrow or umbilical cord tissue, into cartilage-progenitor cells or chondrocytes, and expansion of those cells in vitro.

The term “confluence” refers to cells substantially covering the entire surface of a cell culture vessel. When confluence occurs, cells contact each other through adhesion receptors and the signals from adhesion molecules cause arrest of cell proliferation (contact inhibition), unless the cells are cancer cells.

The term “scaffold” refers to a material that provides mechanical support for cells during transplantation for tissue repair, such as chondrocytes and osteoblasts, endothelial/smooth muscle, skin and other cells or their progenitors.

Intact bone marrow may be obtained by known surgical techniques, as a waste from surgical procedures. It may be aspirated from bone by standard means known to those of skill in the art. Intact umbilical cord tissue may be obtained from umbilical cord by standard means known to those of skill in the art.

Intact bone marrow or intact umbilical cord tissue may be obtained from a human or a non-human source. If human, the source of the intact bone marrow or intact umbilical cord tissue may be autologous or allogeneic from the standpoint of subsequent use, e.g., transplantation of cells produced by the inventive methods.

The present invention provides for a method of generating and expanding tissue-progenitor cells or mature tissue cells in culture, comprising culturing intact bone marrow or intact umbilical cord tissue in a cell differentiation medium whereby tissue-progenitor cells or mature tissue cells are generated from mesenchymal stem cells (MSCs)/progenitor cells present in the intact bone marrow or intact umbilical cord tissue, and expanded.

MSCs/progenitor cells present in intact bone marrow or intact umbilical cord tissue can be differentiated into numerous cell types by the selection of an appropriate differentiation medium. Generally, differentiation medium for culturing and differentiation of stem cells into different cell types is well known in the art.

Osteogenic differentiation medium for differentiation of intact bone marrow or intact umbilical cord tissue into bone-progenitor cells or more mature bone cells (osteoblasts, osteoclasts, and osteocytes) typically contains a cell culture medium, a corticosteroid and a reducing agent. In some embodiments of the invention, the osteogenic differentiation medium contains β-glycerophosphate, L-ascorbic acid-2-phosphate, dexamethasone and either bovine or human serum. In some embodiments of the invention, the osteogenic differentiation medium contains basic fibroblast growth factor FGF and other growth factors or a cytokine.

In some embodiments of the invention, the intact bone marrow or intact umbilical cord tissue is cultured until the cells acquire osteoblast morphology or expression of osteoblast-specific genes and proteins. Examples of such osteoblast-specific genes includes RUNX-2 transcription factor, bone-specific alkaline phosphatase, procollagen aminoterminal propeptide, type I collagen, osteopontin, bone sialoprotein, osteocalcin, parathyroid hormone receptor, osteoprotegerin and receptor activator NF-KB ligand (RANKL). In some embodiments of the invention, the intact bone marrow or intact umbilical cord tissue is cultured until bone tissue-progenitor cells are capable of further differentiation into osteoblasts or mature osteocytes as confirmed by an increase in alkaline phosphatase (ALP) activity and calcium deposition. In some embodiments, the osteogenic differentiation medium is an osteoclast differentiation medium for differentiation of the intact bone marrow or intact umbilical cord tissue into osteoclast progenitor cells and their expansion. In some embodiments of the invention, the osteoclast differentiation medium contains a cell culture medium such as α-MEM, vitamin D₃ and RANKL.

Neurogenic differentiation medium for culturing and differentiation of the intact bone marrow or intact umbilical cord tissue into neuronal progenitor cells typically contains a cell culture medium, a corticosteroid and a reducing agent. In some embodiments of the invention, the neurogenic differentiation medium contains a cell culture medium such as DMEM/F12 (1:1) medium, neurobasal medium or other common cell culture media, β-mercaptoethanol, MEM non-essential amino acids, basic fibroblast growth factor (FGF), epidermal growth factor (EGF), nerve growth factor (NGF), brain-derived growth factor (BDGF), neurotrophin-3, N2, B27 supplements, insulin, transferrin, selinate, dimethylsulfoxide (DMSO), butylated hydroxyanisole (BHA), all-trans retinoic acid (RA), forskolin, valproic acid and KCl.

In some embodiments of the invention, the intact bone marrow or intact umbilical cord tissue is cultured until the tissue-progenitor cells or mature tissue cells acquire neuroblast morphology or expression of neuroblast-specific genes and proteins such as nestin and poly-syalilated-neural cell adhesion molecule (PSA-NCAM). In some embodiments of the invention, the intact bone marrow or intact umbilical cord tissue is cultured until the tissue-progenitor cells are capable of further differentiation into mature tissue cells, such as neurons, as confirmed by increase of neuronal marker expression such as neuronal β-tubulin and neuron-specific enolase. In some embodiments of the invention, the tissue cells exhibit neuron-specific morphology comprising presence of long axons and dendrites, thus confirming the generation and expansion of neurons.

Endothelial differentiation medium for differentiation of the intact bone marrow or intact umbilical cord tissue into vasculature/endothelial-progenitor cells and for their expansion typically contains a cell culture medium, a corticosteroid and growth factors. In some embodiments of the invention the endothelial differentiation medium contains VEGF, FGF, IGF-1 and IGF-2, EGF and hydrocortisone.

Adipogenic differentiation medium for differentiation of the intact bone marrow or intact umbilical cord tissue into adipocyte progenitors or mature adipocytes and for their expansion typically contains a cell culture medium, insulin, and 3-isobutyl-methylxanthine. In some embodiments of the invention, the adipogenic differentiation medium contains dexamethasone, 3-isobutyl-1-methylxanthine, insulin, and indomethacin.

Cardiomyogenic differentiation medium for differentiation of the intact bone marrow or intact umbilical cord tissue into heart muscle progenitor cells or mature cardiomyocytes and for their expansion typically contains a cell culture medium and 5-azacytidine. In some embodiments of the invention, the cardiomyogenic differentiation medium contains bFGF, human and/or bovine serum, and 5-azacytidine.

Pancreogenic differentiation medium for differentiation of the intact bone marrow or intact umbilical cord tissue into progenitors of pancreatic β-cells or into mature pancreatic P-cells and for their expansion typically contains a cell culture medium such as RPMI-1640, low or high glucose DMEM, or N2 medium, nicotinamide. In some embodiments of the invention, the pancreogenic differentiation medium contains nicotinamide, β-mercaptoethanol, exendin 4, activin, B27, bFGF, IGF-1 or IGF-2.

Chondrogenic differentiation medium for differentiation of the intact bone marrow or intact umbilical cord tissue into cartilage progenitor cells or mature chondrocytes and for their expansion typically contains a cell culture medium such as high glucose DMEN and TGF-β3. In some embodiments of the invention, the chondrogenic differentiation medium contains TGF-β3, ascorbic acid, insulin-transferrin-selenate mixture, non-essential amino-acids, proline, glutamine and a corticosteroid.

More specifically, examples of differentiation medium for use in the current invention are set forth in Tables 1-11.

An example of a composition of an osteogenic differentiation medium used in accordance with the present invention is presented in Table 1.

TABLE 1
Cell culture medium includes, but is not limited to,
α-MEM, DMEM, or other common medium.

| COMPONENTS | CONCENTRATION RANGE |
|---|---|
| FCS, heat inactivated or not | 0-10% |
| β-Glycerophosphate | 0 μM-50 mM |
| L-ascorbic acid-2-phosphate | 0.5 μM-0.5 mM |
| (Mg salt n-hydrated) | |
| Dexamethasone (added freshly | 10-1000 nM |
| at each feeding) | |
| Penicillin | 0-100 units/ml |
| Streptomycin | 0-0.1 mg/ml |
| Amphotericin B | 0-25 mg/ml |

An example of a composition of an endothelial differentiation medium used in accordance with the present invention is presented in Table 2.

TABLE 2
Cell culture medium includes, but is not limited to,
low glucose DMEM, MCDB-131, 199 medium, EGM-2
or other common medium.

| | CONCENTRATION | |
|---|---|---|
| COMPONENTS | RANGE | |
| FCS | 0-20% | |
| VEGF | 5-100 | ng/ml |
| Basic FGF | 0-20 | ng/ml |
| EGF | 0-100 | ng/ml |
| IGF-1 | 0-100 | ng/ml |
| Hydrocortisone | 1-10 | μg/ml |
| Penicillin | 0-100 | units/ml |
| Streptomycin | 0-0.1 | mg/ml |
| Amphotericin B | 0-25 | mg/ml |

When bone marrow was incubated for up to 3 weeks in the presence of VEGF, FGF, EGF and hydrocortisone, cells expressing endothelial lineage surface markers, such as Flk-1, Flt-1, VE-Cadherin, vWF, CD105 could be observed. Cells bind endothelial cell specific agglutinin Ulex and accumulate ac-LDL.

An example of a composition of a neurogenic differentiation medium used in accordance with the present invention is presented in Table 3.

TABLE 3
Cell culture medium includes, but is not limited to,
DMEM, DMEM/F12, neurobasal medium (N5), N2
or other common medium.

| | CONCENTRATION | |
|---|---|---|
| COMPONENTS | RANGE | |
| FCS, heat inactivated or not | 0-20% | |
| β-mercaptoethanol | 0-0.5% | |
| 1% MEM non-essential amino | 0-5% | |
| acids | | |
| Glucose | 0-5% | |
| insulin | 5-50 | mg/L |
| Apo-transferrin | 5-100 | μg/ml |
| Sodium selenate | 5-50 | nM |
| Progesterone | 0-50 | nM |
| Putrescine | 0-100 | μM |
| Sodium bicarbonate | 0-5 | Mm |
| HEPES | 0-10 | mM |
| Heparin | 0-5 | μg/ml |
| Basic fibroblast growth factor | 2-20 | ng/ml |
| (bFGF) | | |
| Epidermal growth factor (EGF) | 0-20 | ng/ml |
| Neurotrophin-3 | 0-20 | ng/ml |
| NGF | 0-100 | ng/ml |
| BDNF | 0-20 | ng/ml |
| Dimethylsulfoxide (DMSO) | 0-5% | |
| Butylated hydroxyanisole (BHA) | 0-500 | μM |
| All-trans retinoic acid (RA) | 0-20 | mM |
| Forskolin | 0-25 | μg/ml |
| Valproic acid | 0-5 | mM |
| K252A | 0-10 | nM |
| KCl | 0-30 | mM |
| Penicillin | 0-100 | units/ml |
| Streptomycin | 0-0.1 | mg/ml |
| Amphotericin B | 0-25 | mg/ml |

An example of a composition of an adipogenic differentiation medium used in accordance with the present invention is presented in Table 4.

TABLE 4
Cell culture medium includes, but is not limited to,
DMEM, DMEM/F-12 or other common medium.

| COMPONENTS | CONCENTRATION RANGE |
|---|---|
| FCS | 0-20% |
| Dexamethasone | 10 nM-5 μM |
| 3-isobutyl-1-methylxanthine | 0.1-2 mM |
| Insulin | 1-100 μg/ml |
| Indomethacin | 50-500 μM |

An example of an osteogenic differentiation medium used in accordance with the present invention to generate and expand osteoclast progenitor cells is presented in Table 5.

TABLE 5
Cell culture medium includes, but is not limited to
α-MEM or other common medium.

| COMPONENTS | CONCENTRATION RANGE |
|---|---|
| FCS, heat inactivated or not | 0-10% |
| Beta-Glycerophosphate | 0-50 mM |
| L-ascorbic acid-2-phosphate | 0.5 μM-0.5 mM |
| (Mg salt n-hydrated) | |
| Dexamethasone (added freshly | 10-1000 nM |
| at each feeding) | |
| RANKL | 1-100 ng/ml |
| Vitamin D₃ | 0-10⁻⁷ M |
| M-CSF | 0-100 ng/ml |
| Penicillin | 0-100 units/ml |
| Amphotericin B | 0-25 mg/ml |

Examples of compositions of differentiation medium for production of heart muscle progenitor cells or cardiomyocytes used in accordance with the present invention are presented in Tables 6 and 7.

TABLE 6
Cell culture medium includes, but is not limited to,
MesenCult growth medium (Basal Medium for Human Mesenchymal
Stem Cells, StemCell Technologies), including mesenchymal stem
cell stimulatory supplements (StemCell Technologies), or other
common medium.

| COMPONENTS | CONCENTRATION RANGE | |
|---|---|---|
| L-glutamine | 0-2 | mM |
| Penicillin | 0-100 | units/ml |
| Streptomycin | 0-100 | mg/ml |
| Amphotericin B | 0-25 | mg/ml |
| 5-azacytidine | 0.1-10 | mM |

TABLE 7
Cell culture medium includes, but is not limited to,
low glucose DMEM or other common medium.

| COMPONENTS | CONCENTRATION RANGE | |
|---|---|---|
| FBS | 0-20% | |
| HS | 0-10% | |
| Penicillin | 0-100 | units/ml |
| Streptomycin | 0-0.1 | mg/ml |
| Amphotericin | 0-25 | mg/ml |
| 5-azacytidine | 0.1-10 | mM |
| bFGF | 0-10 | mg/ml |

Examples of compositions of differentiation medium for production of progenitors of pancreatic β-cells used in accordance with the present invention are presented in Tables 8-10.

TABLE 8
Cell culture medium includes, but is not limited to,
serum-free high glucose or low glucose DMEM or
other common medium.

| COMPONENTS | CONCENTRATION RANGE | |
|---|---|---|
| β-mercaptoethanol) | 0.1-0.5 | mM |
| Non-essential amino acids | 0-1% | |
| β-fibroblast growth factor | 0-20 | ng/ml |
| (bFGF) | | |
| EGF | 0-20 | ng/ml |
| B27 | 0.1-2% | |
| L-glutamine | 0-2 | mM |
| β-cellulin | 0-10 | ng/ml |
| Activin A | 0-10 | ng/ml |
| Nicotinamide | 0.1-10 | mM |
| Penicillin | 0-100 | units/ml |
| Streptomycin | 0-0.1 | mg/ml |
| Amphotericin | 0-25 | mg/ml |

TABLE 9
Cell culture medium includes, but is not limited to,
L-DMEM, serum-free H-DMEM, or other common medium.

| COMPONENTS | CONCENTRATION RANGE | |
|---|---|---|
| Nicotinamide | 0.1-10 | mM |
| β-mercaptoethanol | 0.1-1 | mM |
| Penicillin | 0-100 | units/ml |
| Streptomycin | 0-0.1 | mg/ml |
| Amphotericin | 0-25 | mg/ml |

TABLE 10
Cell culture medium includes, but is not limited to,
RPMI 1640 medium or other common medium.

| COMPONENTS | CONCENTRATION RANGE | |
|---|---|---|
| FCS | 0-20% | |
| Glucose | 5.5-23 | mM |
| Nicotinamide | 0.1-10 | mM |
| Exendin 4 | 0.1-10 | nM |
| Penicillin | 0-100 | units/ml |
| Streptomycin | 0-0.1 | mg/ml |
| Amphotericin | 0-25 | mg/ml |

An example of a chondrogenic differentiation medium used in accordance with the present invention is presented in Table 11.

TABLE 11
Cell culture medium includes, but is not limited to,
low-glucose DMEM, MCDB-201 medium, or other common medium.

| COMPONENTS | CONCENTRATION RANGE | |
|---|---|---|
| TGF-β3 | 1-100 | ng/ml |
| Insulin | 1-100 | μg/ml |
| Transferrin | 0-10 | μg/ml |
| Selenium | 0-10 | ng/ml |
| BSA | 0-5 | μg/ml |
| Linoleic acid | 1-10 | μg/ml |
| Dexamethasone | 10-1000 | nM |
| Ascorbic acid | 0-1 | mM |
| Platelet derived growth | 0-100 | ng/ml |
| factor | | |
| EGF | 0-100 | ng/ml |
| IGF | 0-20 | ng/ml |
| Leukemia Inhibitory Factor | 10-10,000 | IU |

The components indicated in Tables 1-11 are added to a conventional cell culture medium, such as DMEM, α-MEM, MCDB-131 medium, McCoys 5A medium, Eagle's basal medium, CMRL medium, Glasgow minimal essential medium, Ham's F-12 medium, Iscove's modified Dulbecco's medium, Liebovitz' 1-15 medium, and RPMI 1640 medium. The list is not exhaustive. In the case of the osteogenic differentiation medium of the invention, α-MEM may be used. For differentiation of neuronal progenitors DMEM/F12 medium or Neurobasal medium supplemented with B27 and/or N2 supplements may be used.

In some embodiments of the present invention, the cell differentiation medium further comprises bone marrow plasma (autologous, allogeneic, or xenogenic).

In addition to the components as described above, the cell differentiation media that are used in accordance with the present invention may contain one or more additional components, if necessary. Such additional components can include a growth factor, a cytokine, a scaffold, an extracellular matrix protein (ECM), demineralized bone matrix, horse or human serum, or antibiotics and antifungal agents, including penicillin G, streptomycin sulfate, amphotericin B, gentamycin and nystatin, which can be added to prevent microorganism contamination.

In some embodiments of the invention, the ECM is selected from collagen, fibronectin, vitronectin, and laminin of a human origin. Typically, the ECM is derived from human peripheral blood, bone marrow or umbilical cord blood.

Generally, the scaffold is selected from synthetic polymers, biological polymers of a human origin, ceramics, gels, alginates, nanofibers, mineralized and demineralized bone matrix. More specifically, scaffolds could be made of natural polymers, such as collagen (or demineralized bone matrix, which is mostly collagen I with attached growth factors), hyaluronic acid, fibrin, etc., or scaffolds could be synthetic polymers such as poly-L-lactide, polyglycolide, lactide-glycolide copolymer, caprolactone-lactide copolymer, poly-caprolactone. Scaffolds also could be inorganic such as ceramics, alumina (Al2O3), hydroxyapatite, β-tricalcium phosphate (TCP), which is chemical derivative of hydroxyapatite or corals that could be transformed into hydroxyapatite, and polyurethanes. Scaffolds could combine ceramics and polymers. Finally scaffolds could be nano-scaffolds that are produced by electrospinning of synthetic and natural polymers.

Typically, the conditions for culturing of intact bone marrow or intact umbilical cord tissue comprise a temperature of about 4-37° C., a humidity of atmospheric to 100% humidity, a carbon dioxide level of 0-5% CO₂ and an oxygen level of 1% oxygen to atmospheric level. Culture conditions for differentiation can be optimized by one skilled in the art.

In some embodiments of the invention, the ratio of intact bone marrow or intact umbilical cord tissue to differentiation medium is between 1:1 and 1:50. Typically, the ratio is 1:6.

Generally, the intact bone marrow or intact umbilical cord tissue is cultured for a period of incubation between 2 and 45 days. In some embodiments of the invention, the period of incubation is 14 days. Typically, the intact bone marrow or umbilical cord tissue is cultured until the tissue-progenitor cells or mature tissue cells become confluent.

Tissue progenitor cells and/or mature tissue cells cultured on culture ware may be harvested by methods known in the art. Generally, the cultured cells are released from the surface to which they are adhered and concentrated by centrifugation. The cells may then be further cultured or used for transplant. Typically, cells are released from the surface to which they are adhered by treatment with a proteolytic enzyme, e.g. trypsin, or by treatment with EDTA.

If cultured on a scaffold, the cells are typically harvested by washing with PBS and harvesting the combined scaffold and cells.

Aside from other advantages disclosed herein, a further advantage of the present invention is due to the differentiation process occurring in a natural environment. AMSCs are thought to come from non-hematopoietic tissue of the bone marrow, referred to as stromal cells. Hematopoietic cells adhere to stromal cells and receive regulatory signals for proliferation and differentiation through adhesion receptors. In addition, stromal cells release soluble factors that activate proliferation and differentiation of hematopoietic cells (Yin and Li, 2006). These interactions between stromal cells and hematopoietic cells are reciprocal. For example, it was recently found that oncostatin M, a factor produced by hematopoietic cells, induces proliferation of human AMSCs and regulates their differentiation (Song et al., 2005, Yanai and Obinata, 2001). Stromal cells also maintain their own growth via autocrine mechanisms. In addition, multiple blood vessels penetrate through stromal niches and provide nourishment to hematopoietic and stromal cells. Commonly used isolation techniques destroy cooperation between various cells of the bone marrow and remove AMSCs from their normal environment. In support of that idea, it was shown that AMSCs derived from single cell suspensions had lower colony formation ability and inferior differentiation potential than AMSC aggregates with megakaryocytes (Miao et al., 2004). Thus differentiation of unprocessed bone marrow occurs within so-called environmental niche, which enhances the differentiation process.

An additional advantage of the present invention is that the method does not require the use of fetal calf serum. Multipotent MSCs have become important tools in regenerative and transplantation medicine. Rapidly increasing numbers of patients are receiving in vitro-expanded MSCs. However, culture conditions for expansion of MSCs typically include fetal calf serum (FSC) because human serum does not fully support growth of human MSCs in vitro. Moreover, only certain lots of FCS are capable of supporting MSC growth. It has been reported that the frequency of the useful lots is 1:30. Besides difficulties in finding of an appropriate lot of FCS, concerns regarding bovine spongiform encephalopathy (BSE), other infectious complications and host immune reactions have fueled investigation of alternative culture supplements. Thus, the use of cellular products for therapy has been generally hindered by the need to include bovine derived sera and/or serum-derived products in the culture media.

Serum-free media for expansion of hematopoietic stem cells and for dendritic cells has already been established. With MSCs, the only published clinical trial reporting the infusion of allogeneic MSCs showed that in one of the patients the possible benefit of the MSCs infusion has been compromised by a lack of engraftment. In this particular patient, an immune reaction against a bovine derived protein was found, suggesting that the use of bovine serum from the earliest phase of cell isolation might be responsible for the immune reaction against MSCs. In contrast, the method of differentiation of intact bone marrow or intact umbilical cord tissue, presented here, could be performed in serum free medium as bone marrow itself is a source of growth factors and cytokines and could produce an effect similar to that of human plasma or autologous serum. Similar techniques could be used for obtaining progenitors of chondrocytes, endothelial cells, cells of various neural lineages, pancreatic β-cells, hepatocytes and skin cells and other progenitors committed to other phenotypes

Because MSCs can differentiate into different cell types, cells differentiated from MSCs can be used to treat many kinds of diseases and conditions. The differentiated cells may be genetically manipulated, e.g., transformed with exogenous nucleic acid, and thus provide gene therapy to the affected or diseased tissue. For example, in addition to the bone injuries and diseases described above, wound healing usually results in scarring, which is caused by the incomplete restoration of initial skin structure and the disruption of the normal alignment of collagen fibers. Moreover, there are specific illnesses and diseases which can result in skin wounds and injuries, such as diabetes ulcers and other ulcerous wounds.

The muscular cardiac tissue is made of cardiomyocytes. These specialized forms of muscle cells are not capable of regeneration following injury in the adult. Common injuries to the heart muscle occur in ischemic heart attacks during which blood flow to the heart is restricted and the cardiac muscle is damaged through hypoxia. Patients suffering from heart infarct require both the restoration of blood supply to the heart and the regeneration of the damaged heart muscle.

The central nervous system, composed of neurons and other neural cells, is generally incapable of regeneration in the adult. The peripheral nervous system is only capable of limited regeneration. Illnesses that commonly result in central nervous system damage are multiple sclerosis and amyotrophic lateral sclerosis. Incidents that commonly result in central nervous system damage are spinal cord damage and cerebral vascular accidents.

Urinary incontinence can result from damage to the sphincters of the urethra. Various conditions can result in liver damage including viral hepatitis, cirrhosis, steatohepatitis and liver cancer.

Similarly damage and degeneration of the pancreas can result in diabetes. Arthritis is a form of degradation and damage to the joints between bones.

Thus, MSCs/progenitor cells can be employed in therapies to improve these conditions, whether the result of bone damage or disease, a disease or the natural imperfection of skin-healing, the inability of heart muscle tissue, nervous tissue, cartilage or joints, liver or urethral sphincters to regenerate, or from diabetes caused by the degeneration of the pancreas.

To treat such diseases and conditions, the tissue-progenitor cells and mature tissue cells generated and expanded in the methods described above, are harvested and transplanted into a patient in need thereof typically by grafting or injecting them at the site of damage or disease. The tissue-progenitor cells or tissue cells may be autologous, allogeneic, or xenogenic.

In the case of a bone defect, the bone progenitor cells are cultured on a scaffold (and directly transplanted into a patient without re-plating), or if cultured on culture ware, may be seeded onto a scaffold after harvesting. The scaffold including the cells is then grafted into the bone defect and secured by known means. In the case of significant bone loss, the scaffold serves as void filler and also provides support for in-growth of host's bone cells, and blood vessels. The scaffold also provides mechanical (as carrier) and biological support for transplanted cells.

In an example of transplantation by injection, neuronal progenitor cells or mature neurons generated and expanded by the methods of the invention may be used to repair or regenerate damaged or diseased nerve tissue. Typically, the harvested cells are suspended in basal medium and injected at the site of the disease or damage. Likewise, cartilage progenitor cells may be transplanted on a scaffold or by intra-articular injection into a patient having cartilage-related, joint damage.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### Cell Culture

Cell culture dishes (6 cm, NUNC) were pre-coated with 10 μg/ml fibronectin (Biological Industries, Israel cat.# 03-090-1) for 2 hours at room temperature, washed twice with PBS and filled up with 5 ml osteogenic differentiation medium pre-warmed at 37° C. [α-MEM/10% FCS/10 mM glycerophosphate/0.2 mM L-ascorbic acid 2-phosphate (Mg salt n-hydrated)/10 nM dexamethasone (added freshly at each feeding)/100 units/ml penicillin/0.1 mg/ml streptomycin 0.25 mg/ml amphotericin B]. Then, 1 ml of the WBM was added to the plate (usually corresponds to 20−50×10⁶ bone marrow cells; cell number varies from donor to donor). The culture remained in the incubator for 2 weeks at 37° C., 100% humidity and 5% CO₂. After one week, half of the medium was replaced with fresh medium without disturbing the cells.

Three variations of this protocol were performed simultaneously:
1. 1 ml bone marrow (containing about 20-50×10⁵ bone marrow cells) was plated into commercial mesenchymal stem cell growth medium instead of osteogenic differentiation medium (Cambrex; Cat. # PT-3001) and placed into a cell incubator for 2 weeks.
2. 1 ml bone marrow (containing about 20-50×10⁶ bone marrow cells) was plated into growth medium (Cambrex; Cat. # PT-3001) and placed into a cell incubator for 1 week. After one week, growth medium was replaced with osteogenic differentiation medium and cells were grown for another week in the cell incubator.
3. 1 ml bone marrow (containing about 20-50×10⁶ bone marrow cells) was plated into osteogenic differentiation medium and placed into a cell incubator for 2 weeks. In addition adult MSCs were isolated from bone marrow according to the commonly used protocol, expanded for two weeks in growth medium and tested in proliferation/differentiation assays (see bellow) along with above described cells for comparison. MSC isolation protocol was as follows: bone marrow sample diluted 1:1 with PBS was layered on Lymphocyte Separation Medium (Ficoll plus sodium diatrizoate salt at 1.077 to 1.080 g/ml) and centrifuged at a low speed for a short time. Mononuclear cells (MNC) were collected, washed and plated onto a fibronectin-coated dish at approximately 10×10⁵ cells/dish (usually cell number was matched to the number of MNC cells in 1 ml bone marrow) in 5 ml commercial growth medium. After 48 hours of incubation in the tissue culture incubator, non-adherent cells were washed out and the remaining adherent cells (presumably MSC) were allowed to expand in growth medium for the rest of the 2 week period with regular feeding every 3-4 days.

At the end of 2 weeks, all of the dishes including those with MSCs generated by conventional method were washed twice with PBS trypsinized and re-plated onto a 24 well plate at 3000 cells/cm² in osteogenic differentiation medium for proliferation and differentiation assays. At days 1, 7, and 14, cell number/well was measured with Calcein-AM assay for viable cells. At the same time, alkaline phosphatase (ALP) activity in differentiating cells was quantitated. At the end of two weeks, formation of calcium nodules outside of the cells (mineralization) was assessed in a quantitative Ca⁺⁺ assay.

Calcein-AM Proliferation Assay

Cells growing in 24 well plates were washed once with PBS and then incubated with 0.5 ml of a 5 μM solution of Calcein-AM (Invitrogen/Molecular Probes Cat# C1430) in phenol-free medium at 37° C. for 30 minutes. Fluorescence of live cells was read on Synergy-BioTek plate reader at excitation/emission of 485/530 nm.

Proliferation of Osteoprogenitors Obtained from Intact Bone Marrow.

As it is well known that bone marrow from different donors varies in terms of AMSC quantity and their differentiation potential, the experiments were performed with two bone marrow samples. One sample was commercial bone marrow aspirate (Cambrex) and the other was surgical bone “waste” that came from the orthopedic department of Hadassah Hospital (Jerusalem, Israel). In both experiments, unprocessed bone marrow was incubated either with growth medium (GM) or with osteogenic differentiation medium (DM) for two weeks or with GM for one week and with DM for another week as described above. Two alternative protocols were applied for control: bone marrow was incubated with growth medium (GM) for two weeks and with growth medium for one week and with differentiating medium for another week (GM-DM). Osteoprogenitors obtained according to these three protocols were compared to AMSCs isolated according to the commonly used method.

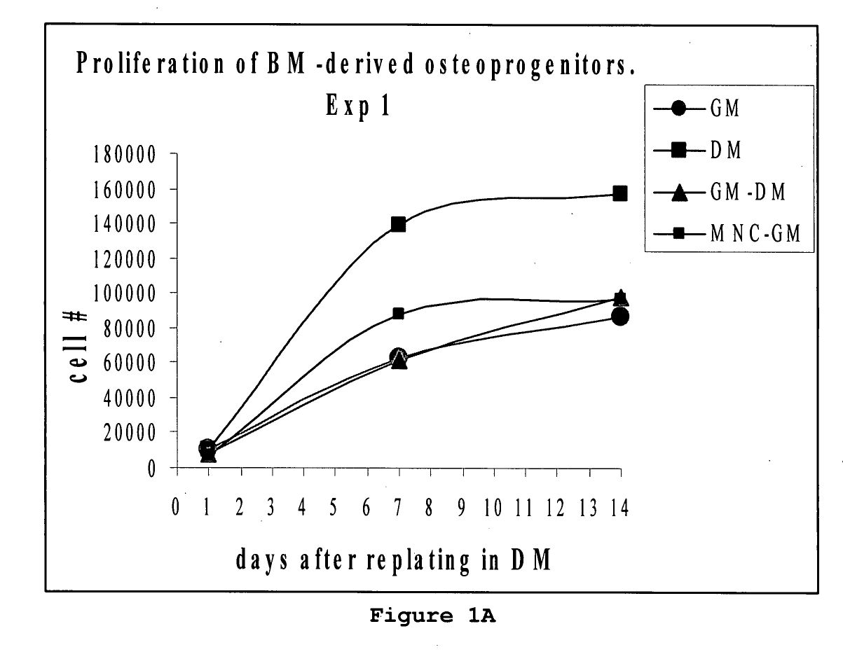

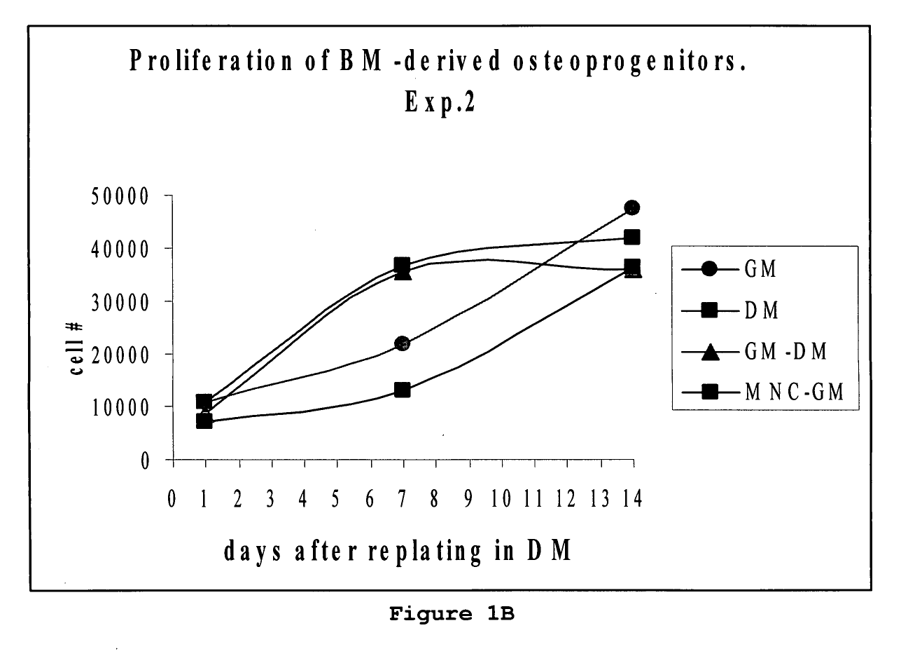

The cells were re-plated onto 24 wells at the identical density (3000 cells/cm²) and allowed to grow in differentiating medium for two weeks at 37° C. Cell number was estimated at day 1, 7 and 14. (According to Calcein assay). The results of this experiment are presented in FIG. 1. Bone progenitors (BP) generated by incubation of BM in DM for 2 weeks, proliferated at a higher rate than cells produced by incubation of BM in GM or in GM-DM. This was true for cells obtained from both, BM aspirate and surgical waste, although all cells produced from the bone marrow aspirate in Experiment 1 proliferated faster than cells produced from the bone marrow surgical sample in Experiment 2 (FIG. 1(A)-Exp. 1 and FIG. 1(B)-Exp. 2). BP in experiment 1 reached maximum proliferation by day 7. At day 7, there were twice as many bone progenitors (BP) generated by incubation of BM in DM as compared to other protocols (FIG. 1(A)-Exp. 1). Similarly, in Experiment 2, BP obtained from incubation in DM achieved higher numbers by day 7 than other cell types. Cells produced by incubation with GM and control MSC grew slower and achieved the maximum proliferation only by day 14 (FIG. 1(B)).

### Example 2

### Measurement of Alkaline Phosphatase (ALP) Activity

Cells were washed twice with PBS and then lysed with 250 μl/well cold lysis buffer [1 mM MgCl₂/0.5% Triton XI00 in Alkaline Buffer Solution (Sigma cat# A9226) and incubated on ice for 1 hour. The reaction mixture of 100 μl cell lysate and 400 μl Phosphatase Substrate Solution (20 mg/ml of p-nitrophenol phosphate (Sigma Cat # N4645) in 5 ml Alkaline Buffer Solution diluted 1:3 with ddH₂O) was incubated at 37° C. for 10 minutes and then returned on ice. The reaction was stopped with 500 μl EDTA-NaOH stop solution (20 g NaOH plus 37.22 g Na₂EDTA in 500 ml ddH₂O). 200 μl of each sample were transferred to a 96 well plate and absorbance was read at 404 nm using Synergy plate reader. The results were expressed as nmol p-NP/ml/min and normalized to the number of living cells in corresponding wells.

ALP activity in osteoprogenitors obtained from intact bone marrow.

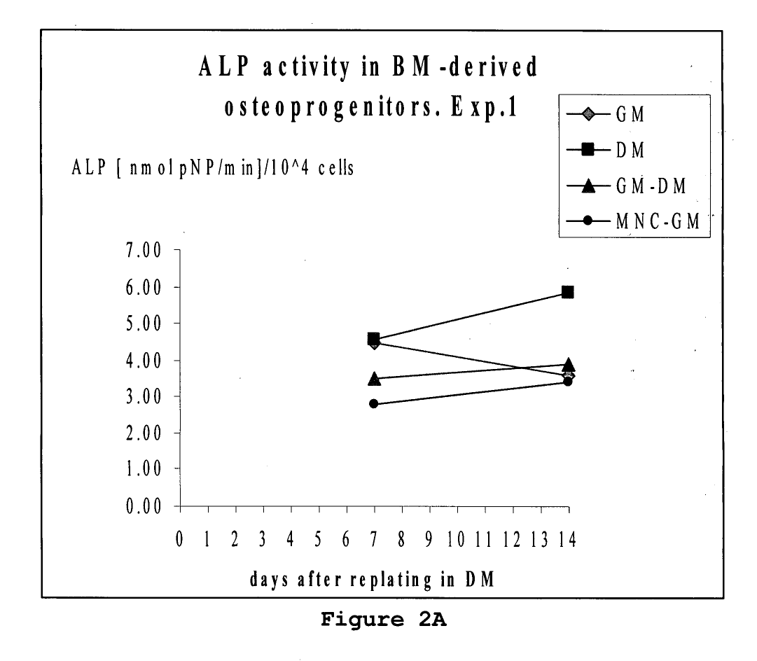

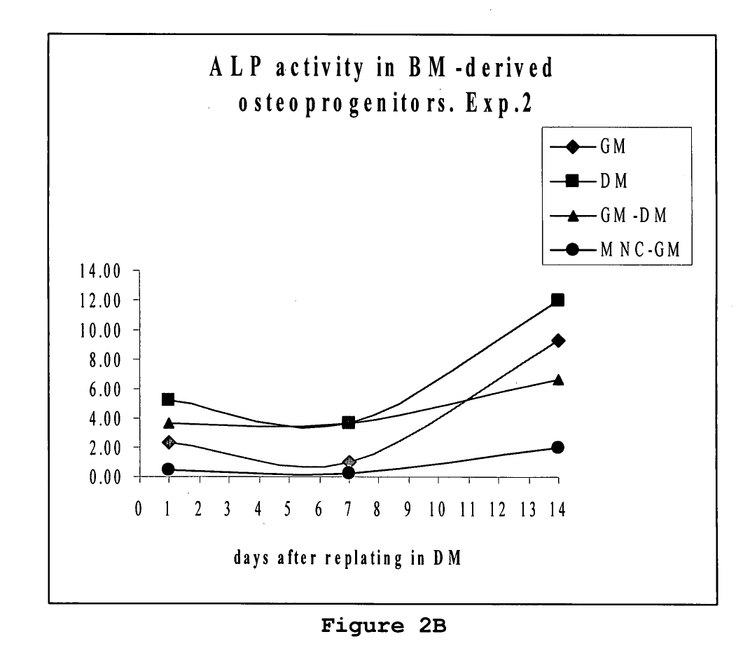

To assess differentiation, BP obtained from the intact bone marrow and control MSC described above were replated in 24 well plates in DM and tested for ALP activity at various times. FIG. 2 demonstrates ALP activity in the produced cultures. In both Experiment 1 and Experiment 2, BP produced by incubation of BM in DM continued to differentiate significantly faster than cells obtained through other protocols (FIG. 2 Exp. 1 and Exp. 2). At 1 week after replating, ALP activity per 10,000 BP was higher than in other cells and further increased at 2 weeks. As shown in Experiment 2, even one day after re-plating, these BP had the highest ALP activity per cell (FIG. 2, Exp. 2). High levels of differentiation of these cells might explain why proliferation of these cells slowed down after day 7, while cells obtained from bone marrow by incubation in the GM or control MSC continued to proliferate after day 7 but did not differentiate as well (FIG. 1(B)-Exp. 2 and FIG. 2(B)-Exp. 2).

### Example 3

### Calcium Deposition Assay

Above mentioned cells grown in 24 well plates with DM were washed twice with PBS and then lysed with 250 μl/well 0.5N HCl. The lysates were shaken at 4° C. overnight to extract calcium and then centrifuged at 1000 rpm for 3 minutes. The assay was set up in 96 well plates using Calcium Liquicolor kit from Stanbio Labs, USA (cat# 0150) according to the manufacturer's instructions. The reaction mixture was incubated for 60 minutes at 37° C. and then absorbance was measured at 550 nm using a Synergy plate reader.

Calcium Deposition in Cultures of BP Obtained from Intact Bone Marrow.

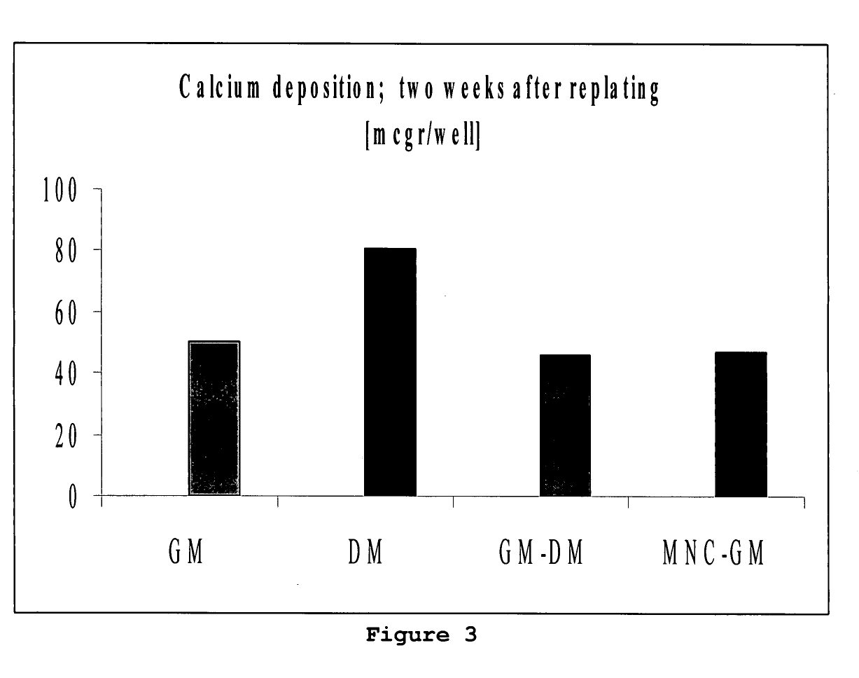

More mature osteoblast progenitors usually lay down extracellular matrix and initiate mineralization by depositing extracellular calcium phosphate. In our experiment, calcium deposition was measured in cultures of BP and MSC at 2 weeks after re-plating onto 24 well plates in differentiating medium as described above. Again, BP produced by incubation of the intact bone marrow in DM deposited more calcium per well than osteoprogenitors produced in other conditions or MSC isolated through adhesion selection (FIG. 3).

### Example 4

### Alizarin-Red Staining of Calcium

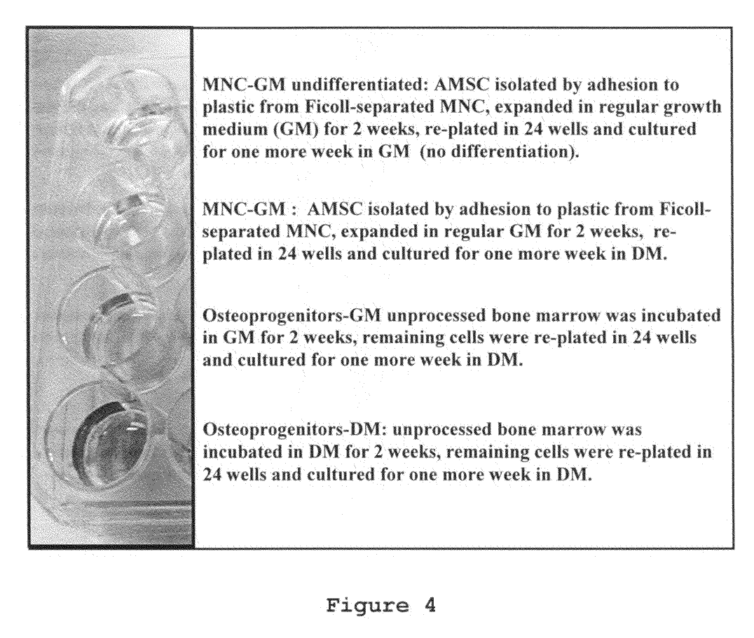

Cultures of BP produced from unprocessed bone marrow and AMSCs were re-plated in a 24 well plate and allowed to differentiate in osteogenic differentiation medium for 1 week. At the end of 1 week, the cultures were fixed in 4% paraformaldehide for 15 minutes at room temperature and then stained for 1 minute with 5 mg/ml alizarin red S solution (Sigma, cat.#A5533) to visualize calcium deposits. The results of this experiment are presented in FIG. 4. In this experiment, control cultures of AMSCs were grown in growth medium (non-differentiating conditions) (top well), and as in previous experiment in osteogenic differentiation medium (second well from the top). No alizarin red staining was observed in these cultures, or in cultures produced by incubation of intact bone marrow in growth medium (3′ well from the top). Only cultures produced by incubation of intact bone marrow with DM contained significant amount of calcium deposits as revealed by bright red staining (bottom well). One week of differentiation was enough for BP to mineralize the whole culture. These cells are better suited to proliferate in the microenvironment of bone injury as they already express receptors and signaling cues of immature bone-producing cells.

### Example 5

### Flow Cytometry Analysis of ALP Expression

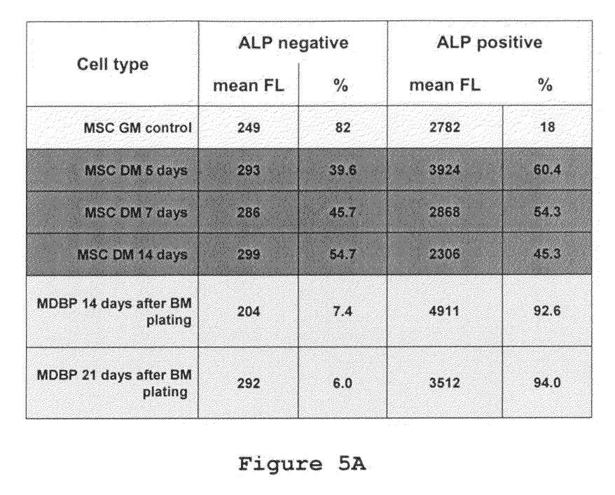

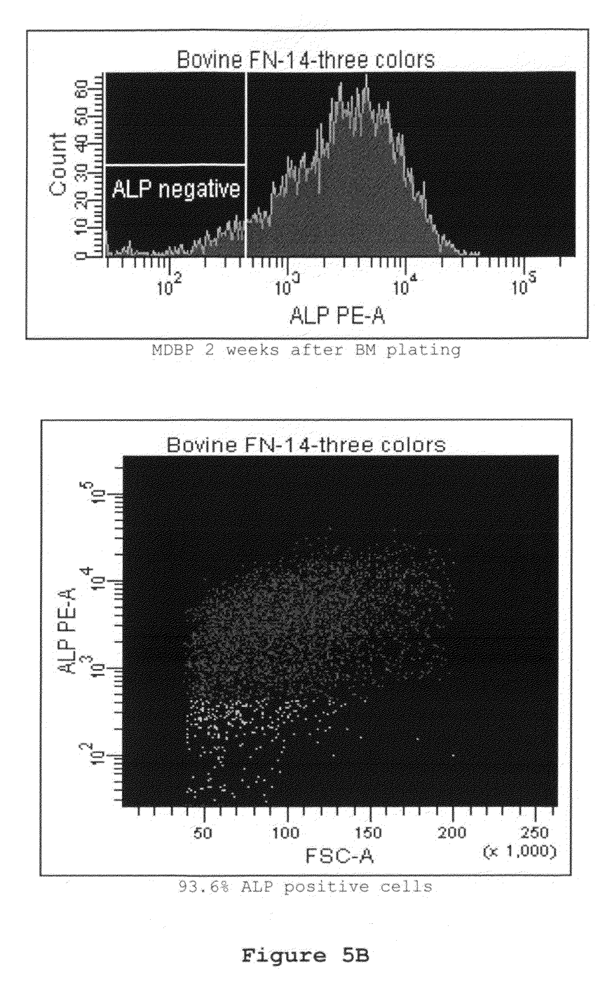

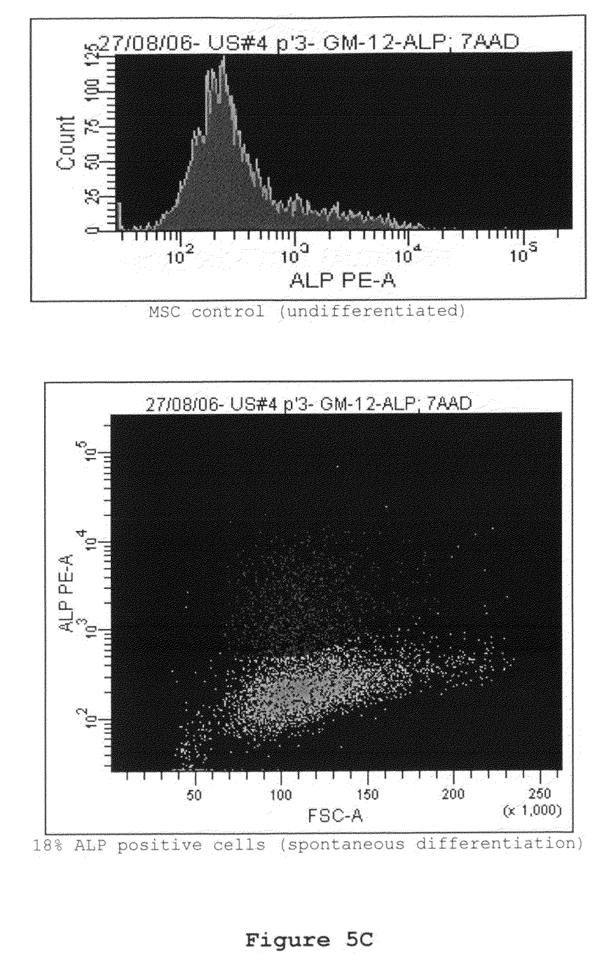

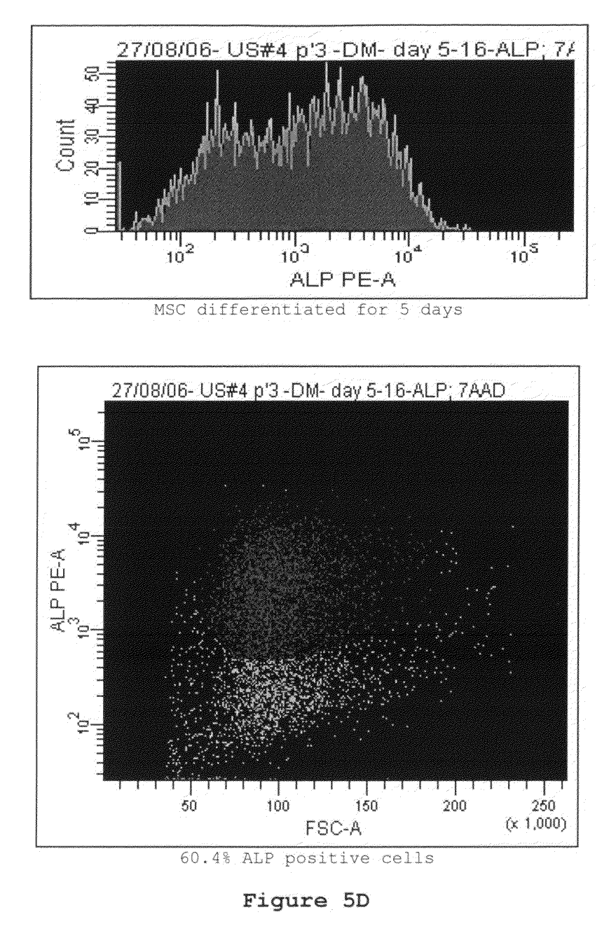

BP were produced by incubation of unprocessed bone marrow with osteogenic differentiation medium for 14 and 21 days and then stained with antibody against bone-specific ALP conjugated to phycoerythrin (PE) (BD cat#556068; clone 1B12) and subjected to FACS analysis using FACSAria flow cytometer (Becton Dickenson). MSCs isolated from bone marrow through conventional adhesion method were incubated in DM for various times and also stained with the same antibody and analyzed on FACSAria for comparison. The statistical analysis results are presented in FIG. 5A.

“Mean Fluorescence” characterizes the number of ALP molecules expressed on the cell membrane. As follows from the table (FIG. 5A), 80% of control undifferentiated MSCs did not express ALP (FIG. 5C). The highest expression of ALP in MSCs undergoing differentiation was observed on day 5 after addition of osteogenic differentiation medium (mean FL 3924). However, only 60% of all cells were ALP positive (FIG. 5D). On dot plots and a histogram presented in FIGS. 5B-C, two populations of MSCs may be seen, one ALP negative and the other ALP-positive. Longer differentiation of MSCs resulted in low levels of expression of ALP and in a decrease in the percentage of ALP positive cells (FIG. 5A). In contrast, more than 90% of bone progenitors produced through differentiation of unprocessed bone marrow for 2 weeks expressed high levels of ALP (mean FL 4911) (FIGS. 5A and B) and remained ALP-positive for an additional 7 days (FIG. 5A). Dot plots and histograms of FIG. 5B confirm homogeneity of bone progenitor population as judged by ALP expression. These results indicate high efficiency of production of bone progenitors with a new method. Thus, the present invention allows for better control of the yield and quantity of bone progenitors for subsequent use in transplantation.

### Example 6a

### Differentiation of Bone Marrow Cells into Neuronal Progenitor Cells

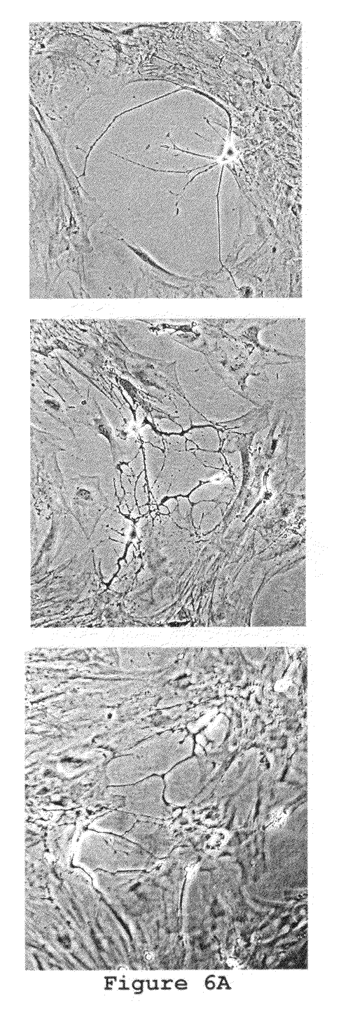

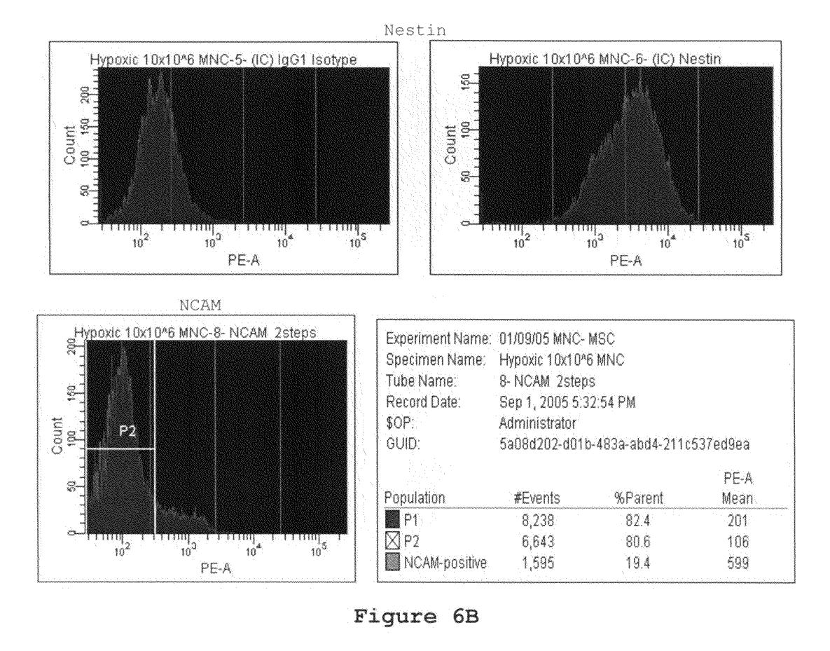

Unprocessed bone marrow was mixed 1:1 with DMEM medium containing 10% FCS, 0.1% β-mercaptoethanol and 1% MEM non-essential amino acids and plated onto culture dishes precoated with fibronectin. Dishes were incubated for two weeks in the cell incubator. After two weeks, the cultures were washed 3 times with PBS. Cells with neuron-like morphology were found during microscopic examination (FIG. 6A). Cells were trypsinized and stained with antibodies against nestin and NCAM, early markers of neuronal differentiation. According to FACS analysis, most of the cells expressed high levels of nestin and almost 20% cells were NCAM-positive (FIG. 6B).

Neural-progenitor tissue derived from MSCs has been transplanted into mice and has shown to differentiate into olfactory bulb granule cells and periventricular astrocytes. (Deng et al. 2006.)

This example shows that cells with markers of neural progenitors (such as nestin and NCAM) can be formed from intact bone marrow using the methods of the present invention. These neural progenitors may be useful in transplantation to form neural tissue in a patient in need thereof.

### Example 6b

### Differentiation of Bone Marrow Cells into Neurons

10 μL of intact bone marrow was incubated with DMEM/F12 (1:1), supplemented with insulin, transferrin, selenate, NaHCO₃, FGF and EGF in a 24 well plate in a cell incubator. The wells were washed on day 4; cells continued to grow in the same medium with addition of putrescine and progesterone. At the end of the incubation, cells were fixed and stained with antibody against neuronal marker class III β-tubulin. As shown in FIG. 6C, all resulting cells were positive and had neuron-specific morphology with long axons and dendrites.

This example shows that neurons can be formed using the differentiation methods of the invention from intact bone marrow.

### Example 7

### Comparison of Cell Yields of Marrow-Derived Bone Progenitors (MDBP) Produced with and without Serum in Cell Culture Plates

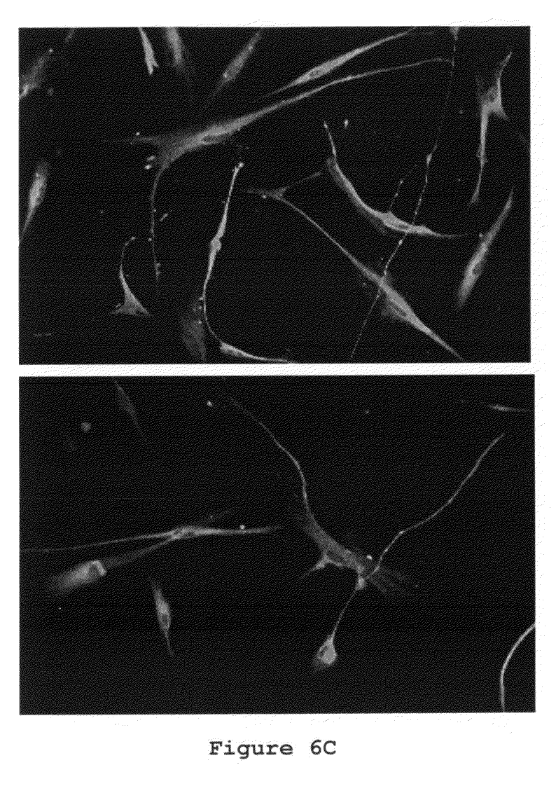

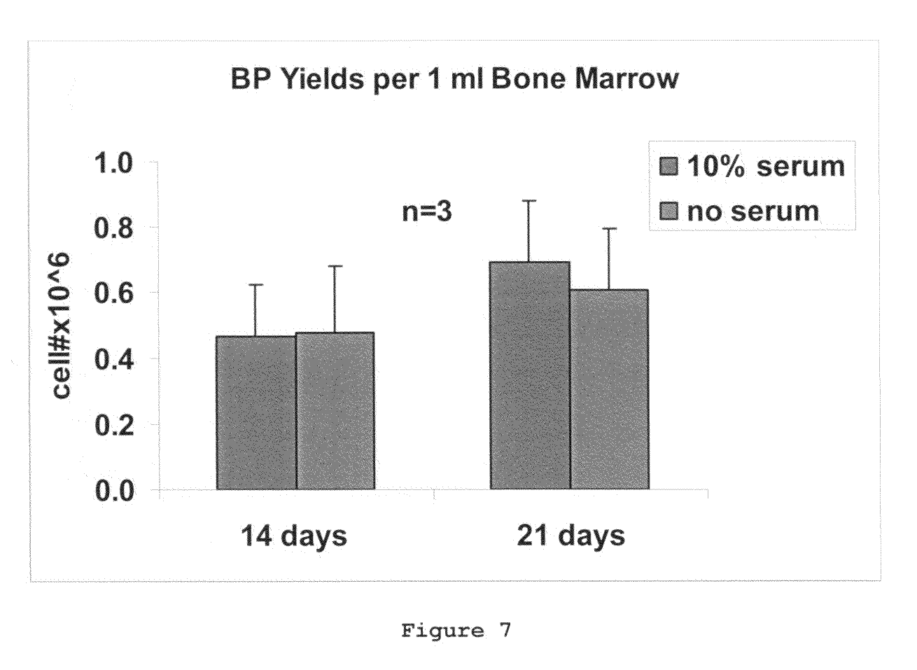

MDBP were produced by culturing of unprocessed bone marrow with osteogenic differentiation medium containing either 10% FCS or no serum at all for 14 and 21 days. At the end of incubation MDBP cultures were washed, cells were detached from the dishes by trypsinization and counted in heamocytometer. In some cases cells were stained with a fluorescent dye Calcein-AM and cell number was determined according fluorescence intensity measured on a plate reader. Cell counts were normalized per volume of the bone marrow added to the culture. No significant differences between cultures with 10% serum and cultures without serum observed (FIG. 7).

### Example 8

### Comparison of Cell Yields of MDBP Produced with and Without Serum on Various Scaffolds

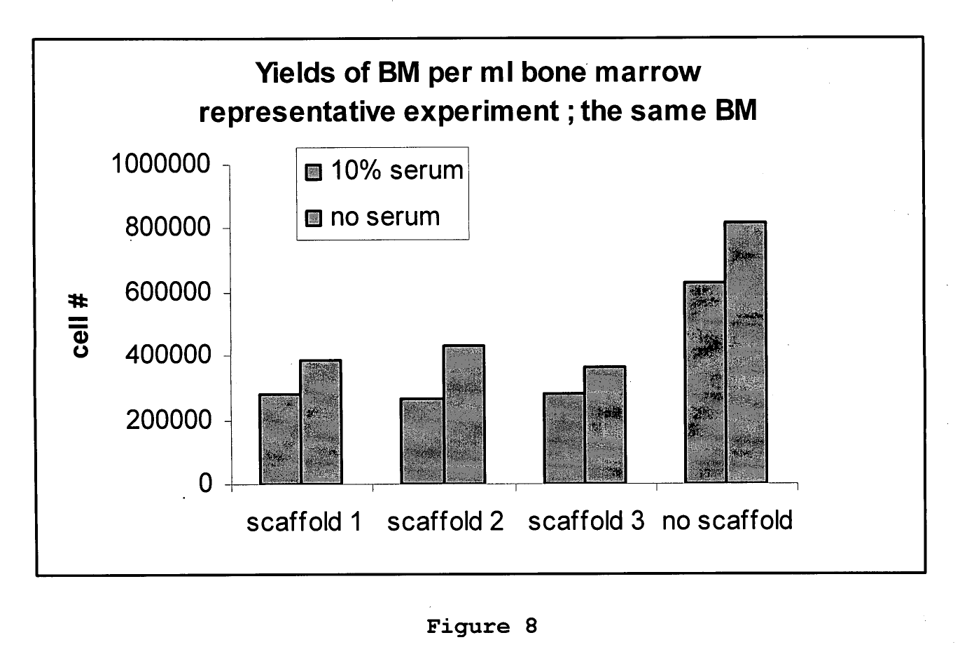

MDBP were produced by culturing of unprocessed bone marrow with osteogenic differentiation medium containing either 10% FCS or no serum for 14 days in the presence of scaffolds of various compositions. At the end of incubation, scaffolds were washed and placed in the medium containing AlamarBlue for 2 hours. Change of AlamarBlue fluorescence that reflects the number of viable cells on a scaffold was measured on a plate reader at Ex/Em 530/590 nm. Cell counts were normalized per volume of the bone marrow added to the culture (FIG. 8).

### Example 9

### Comparison of ALP Activity BP Produced with and without Serum Using Quantitative Assay

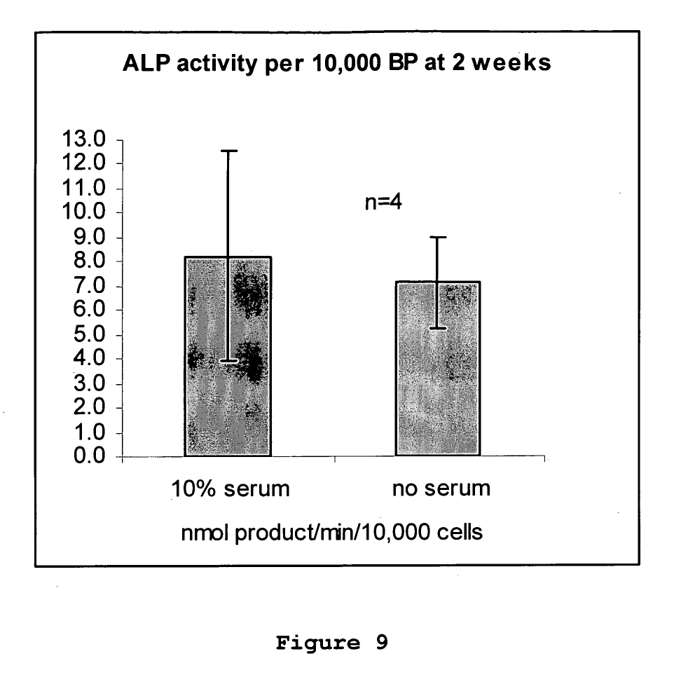

MDBP were produced by culturing of unprocessed bone marrow with osteogenic differentiation medium containing either 10% FCS or no serum in 24 well plates for 14 days. The MDBP cultures were washed with PBS lysed with 250 μl/well cold lysis buffer [1 mM MgCl₂/0.5% Triton X100 in Alkaline Buffer Solution (Sigma cat# A9226)] and incubated on ice for 1 hour. The reaction mixture of 100 μl cell lysate and 400 μl Phosphatase Substrate Solution (20 mg/ml of p-nitrophenol (p-NP) phosphate (Sigma Cat # N4645) in 5 ml Alkaline Buffer Solution diluted 1:3 with ddH₂O) was incubated at 37° C. for 10 minutes and then returned on ice. The reaction was stopped with 500 μl EDTA-NaOH (20 g NaOH plus 37.22 g Na₂EDTA in 500 ml ddH₂O) and 200 μl of each sample was transferred to a 96 well plate and absorbance was read at 404 nm using Synergy plate reader. The results were expressed as amount of p-NP produced per 1 ml lysate per 1 min in each well. No significant differences were observed (FIG. 9).

### Example 10

### Comparison of ALP Activity in MDBP Produced with and Without Serum Using FAST Blue Staining

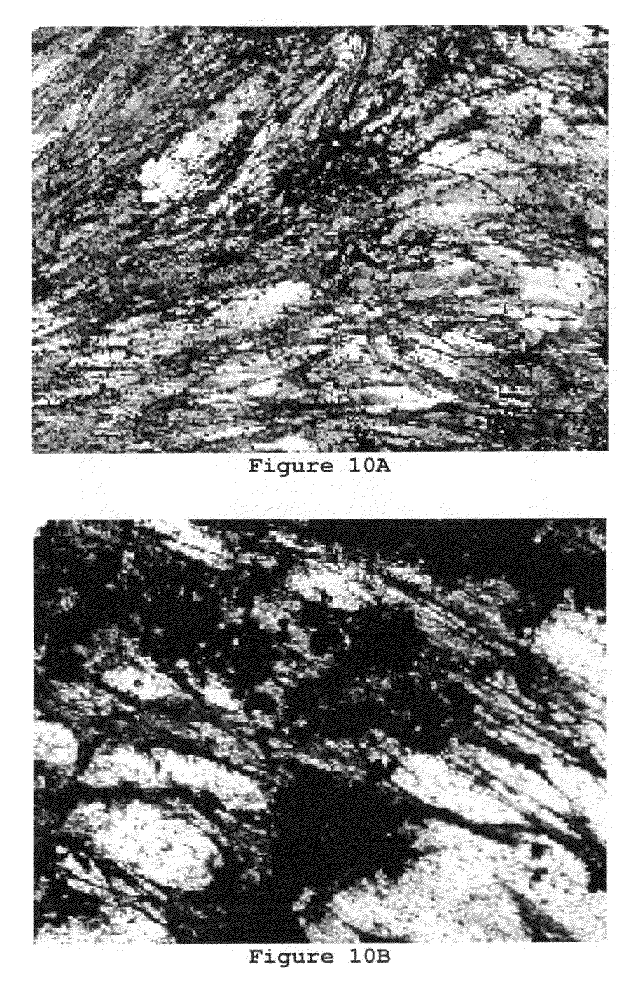

MDBP were produced by culturing of unprocessed bone marrow with osteogenic differentiation medium containing either 10% FCS or no serum at all for 21 days and then washed and fixed in citrate/acetone for 30 sec at room temperature. Cells were then stained for 30 min at room temperature with Naphthol AS-MX phosphate as a substrate for ALP (Sigma, Alkaline Phosphatase Fast Blue Staining Kit; cat# 85-L1). Bone progenitors (BP) produced in medium without serum are positive for ALP similar to control cells produced with 10% serum (FIG. 10).

### Example 11

### Comparison of Surface Expression of Bone-Specific ALP in MDBP Produced with and without Serum

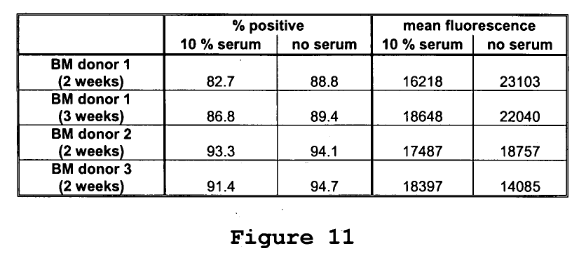

MDBP were produced by culturing of unprocessed bone marrow with osteogenic differentiation medium containing either 10% FCS or no serum at all for 2 weeks and in some experiments for 3 weeks and then stained with antibody against bone-specific ALP conjugated to allophycocyanin (APC) (clone; B4-78; R&D; cat.# FAB1448A). Cells were subjected to FACS analysis using FACSAria flow cytometer (Becton Dickenson). Staining with irrelevant antibody of the same isotype was used as a negative control. Statistical analysis of results presented in FIG. 11 demonstrate that there were no differences in percentage of ALP-positive cells between BP produced with or without serum and that mean fluorescence of cells, which reflects the level of ALP protein on the cell surface was the same or even greater in BP grown without serum.

### Example 12

### Comparison of Mineralization in MDBP Cultures Produced with and without Serum

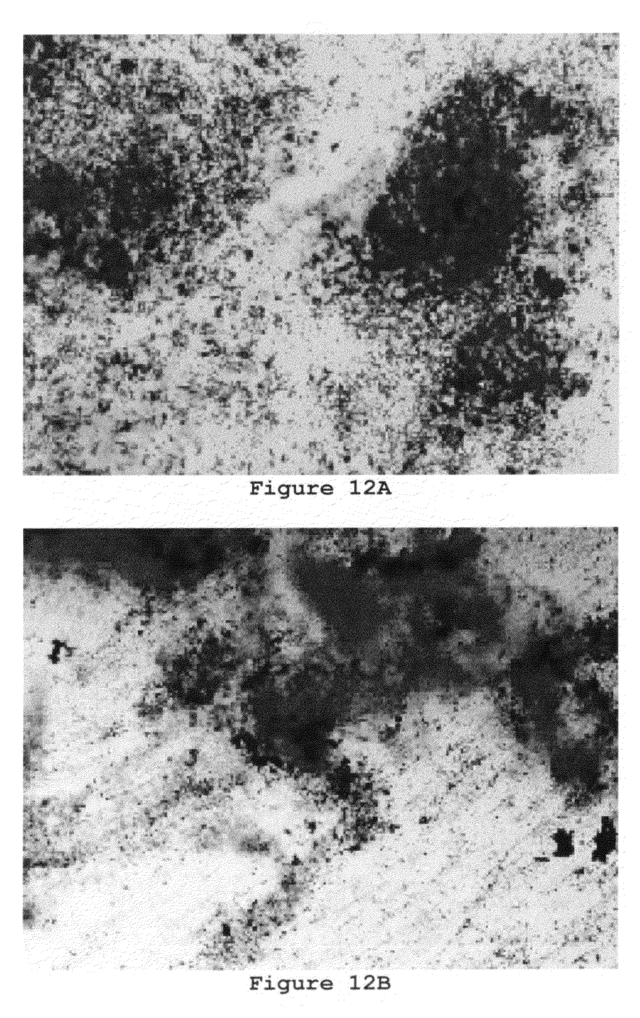

MDBP were produced by culturing of unprocessed bone marrow with osteogenic differentiation medium containing either 10% FCS or no serum at all for 21 days and then washed and fixed in 4% paraformaldehyde for 15 minutes at room temperature and then stained for 1 minute with 5 mg/ml alizarin red S solution (Sigma, cat.#A5533) to visualize calcium deposits. MDBP produced in medium without serum laid down calcium deposits similar to control cells produced with 10% serum (FIG. 12).

### Example 13

### Production of Osteoclasts from Unprocessed Bone Marrow

Osteoclast progenitors were produced by culturing of unprocessed bone marrow with osteogenic differentiation medium containing no serum [α-MEM/10 mM glycerophosphate/0.2 mM L-ascorbic acid 2-phosphate (Mg salt n-hydrated)/10 nM dexamethasone (added freshly at each feeding) 100 units/ml penicillin/0.1 mg/ml streptomycin 0.25 mg/ml amphotericin] supplemented with osteoclast inducing factors [B/RANKL (50 ng/ml)/vitamin D₃ (10⁻⁸M) and M-CSF] for 14 days. At the end of incubation the cultures were washed with PBS, and stained with osteoclast marker, tartrate resistant acid phosphatase, (TRAP) using staining kit (Sigma) according to manufacturer instructions. The results are presented in FIG. 13, and demonstrate a method of production of osteoblast/osteoclast mixed culture at natural ratios. Osteoclasts present in osteoblast culture will improve bone remodeling after transplantation, resulting in stronger new bone.

### Example 14

### Comparison of Surface Expression of Bone-Specific ALP in MDBP Produced on Fibronectin-Coated and Bone Marrow (BM) Plasma-Coated Tissue Culture Plates

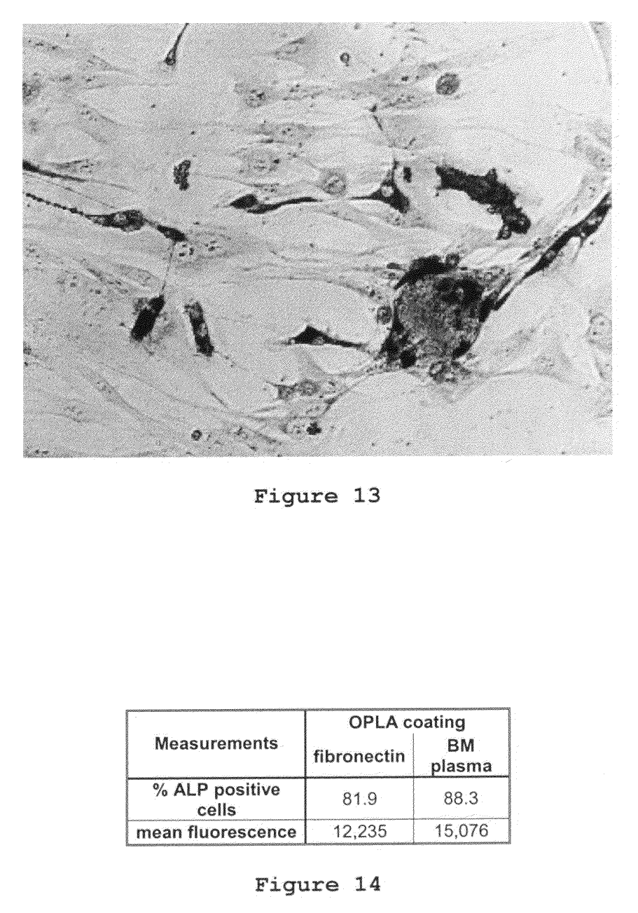

MDBP were produced by culturing of unprocessed bone marrow with osteogenic differentiation medium/no serum in 60 mm tissue culture plates coated either with bovine FN (10 ng/ml for 4 hours at 37° C., washed twice with PBS) or with BM plasma (overnight at 37° C., washed once). At 14 days after the start of the culture Bone Progenitors (BP) were trypsinized and stained with antibody against bone-specific ALP conjugated to allophycocyanin (APC) (clone; B4-78; R&D; cat.# FAB1448A). Cells were subjected to FACS analysis using FACSAria flow cytometer (Becton Dickinson). Staining with irrelevant antibody of the same isotype was used as a negative control. There were no differences in percentage of ALP-positive cells between BP produced on tissue culture plastic coated with fibronectin and with BM plasma (FIG. 14). Similarly, mean fluorescence of cells, which reflects the level of ALP protein on the cell surface, was the same or even greater in BP grown on BM plasma.

### Example 15

### Comparison of ALP Activity of MDBP Produced by Culturing of Unprocessed Bone Marrow on a Scaffold Coated with Fibronectin or with BM Plasma

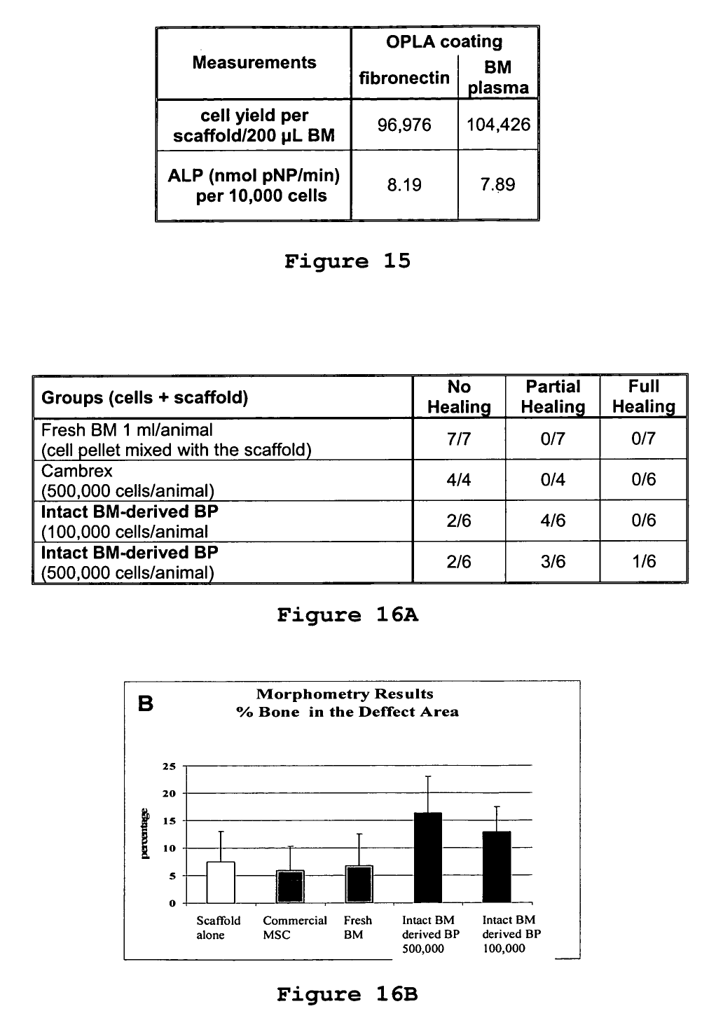

MDBP were produced by rotating unprocessed bone marrow with osteogenic differentiation medium/no serum in the presence of OPLA scaffolds (Beckton Dickinson, cat #354614). The scaffolds were coated either with bovine FN (10 ng/ml for 4 hours at 37° C., washed twice with PBS) or with BM plasma (overnight at 37° C., washed once). At 14 days after the start of the culture, the scaffolds were washed with PBS, the number of adherent cells was measured with a Trypan Blue assay and then the cells were lysed with 250 μl/well cold lysis buffer mM MgCl₂/0.5% Triton X100 in Alkaline Buffer Solution (Sigma cat# A9226)] and incubated on ice for 1 hour. The reaction mixture of 100 μl cell lysate and 400 μl Phosphatase Substrate Solution (20 mg/ml of p-nitrophenol (p-NP) phosphate (Sigma Cat # N4645) in 5 ml Alkaline Buffer Solution diluted 1:3 with ddH₂O) was incubated at 37° C. for 10 minutes and then returned on ice. The reaction was stopped with 500 μl EDTA-NaOH (20 g NaOH plus 37.22 g Na₂EDTA in 500 ml ddH₂O) and 200 μl of each sample were transferred to a 96 well plate and absorbance was read at 404 nm using Synergy plate reader. The results were expressed as amount of p-NP produced per 10,000 per 1 min. No significant differences were observed between BP grown on fibronectin-coated OPLA scaffolds and those grown on BM plasma-coated scaffolds (FIG. 15).

### Example 16

### In Vivo Transplantation of Human BM-Derived BP in Critical Size Femoral Defect Model in Nude Mice

BP were derived from intact human bone marrow according to Example 1 and put on a hydrogel-ceramic scaffold. The scaffolds with cells were transplanted into nude mice at the site of femoral bone defect at two doses: 100,000 cells per defect and 500,000 per defect. The defect size was 3 mm, which is considered a critical size defect as it does not heal by itself. Control groups of animals were transplanted a) with scaffold alone, b) with fresh BM-derived cell pellet mixed with the scaffold; c) with commercial undifferentiated MSCs derived from human BM through common method of adhesion selection, seeded on the scaffold.

FIG. 16 presents the results of X-ray evaluation (FIG. 16A) and of morphometric evaluation of histological sections (FIG. 16B) performed at the end of the study at 8 weeks after transplantation. According to X-ray tests partial or even full bone healing (one animal in animal group transplanted with 500,000 BP) was observed only in animals transplanted with BP derived from intact BM according to the described method. No bone healing was observed in animals transplanted with commercial MSCs or with fresh BM-derived pellet (FIG. 16A).

At the end of the study bones were demineralized and paraffin embedded sections of the defect area were stained with Hematoxylin and Eosin (H&E) for general morphology and with Masson's Trichrome for the visualization of bone, connective tissue and blood vessels. Sections were microphotographed using a digital camera, and the amount of the new bone formed in the defect area was measured using morphometric software. The results demonstrate that only BP produced with described method helped to increase new bone formation up to 15-20% of the defect area, while commercial MSCs and fresh BM pellet had no effect (FIG. 16B).

This example shows that tissue progenitor cells formed using the method of the invention can be transplanted into patients in need of such therapy.

### Example 17

### Obtaining Differentiated Cells From Umbilical Cord Wharton's Jelly (Matrix)

Umbilical cords are obtained from local maternity hospitals after normal deliveries, with approval by institutional review board (Helsinki). Umbilical cord segments 1-3 cm in length are cut longitudinally to expose the two umbilical arteries and the umbilical vein. The vessels are removed and discarded. The remaining umbilical cord tissue including the Wharton's jelly is diced into 2-5 mm³ explants using single edge razor blades, transferred to 2-10 ml of osteogenic, endothelial, chondrogenic or neurogenic differentiating medium and plated onto ECM or scaffold for 10-21 days. The resulting cells are examined for specific cell markers as described above.

Alternatively:

Collagenase type I (1-2 mg/ml) is added to the Wharton's jelly-differentiating medium mixture for 1-16 hours at 37° C. to loose tissue connections. The action of the enzyme is stopped by collagenase inhibitor and incubation of tissue in differentiating medium is continued for 10-21 days in the presence of the scaffold and/or ECM.

Plasma obtained by centrifugation of umbilical cord blood (UCB) or maternal blood at 1000×g for 10 min could be added to differentiating medium at ratio 1:2 to 1:20 to substitute for FCS. Plasma could be stored at 4° C. or frozen at minus 20° C. for consecutive feedings of resulting progenitor cells.

### Example 18

### Obtaining Differentiated Cells from Umbilical Cord Blood

Umbilical cord blood (UCB) is mixed in a ratio of between 1:2 and 1:10 with osteogenic, chondrogenic, endothelial or neurogenic differentiating medium and plated onto ECM or scaffold for 10-21 days. The resulting cells are examined for specific cell markers as described above.

Fetal calf serum could be omitted from differentiating medium. Part of UCB is set aside for production of UCB plasma. UCB plasma is obtained by centrifugation of UCB at 1000×g for 10 min and could be added to differentiation medium at ratio 1:2 to 1:20 to substitute for FCS and used for further feedings of resulting progenitors. Plasma is stored at 4° C. or frozen at minus 20° C.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### REFERENCES

Aslan H, Zilberman Y, Kendel A, Liebergall M, Oskouian R J, Gazit D, Gazit Z. Osteogenic Differentiation of Noncultured Immunoisolated Bone Marrow-Derived CD105+ Cells. Stem Cells. 2006
Bedada F B, Gunther S, Kubin T, Braun T. Differentiation versus plasticity: fixing the fate of undetermined adult stem cells. Cell Cycle. 2006 February; 5(3):223-6.
Bianco P., Robey P. G., Marrow stromal stem cells, J. Clin. Invest., 105: 1663-1668, 2000
Chen J, Chopp M. Neurorestorative treatment of stroke: cell and pharmacological approaches. NeuroRx. 2006 October; 3(4):466-73. Review.
Caplan A I. Review: mesenchymal stem cells: cell-based reconstructive therapy in orthopedics. Tissue Eng. 2005 July-August; 11(7-8):1198-211
Colter D. C., Sekiya I., Prockop D. J., Identification of a subpopulation of rapidly self-renewing and multipotential adult stem cells in colonies of human marrow stromal cells, Proc. Natl. Acad. Sci. USA, 98: 7841-7845, 2000
Deng J, Petersen B E, Steindler D A, Jorgensen M L, Laywell E. D., Mesenchymal stem cells spontaneously express neural proteins in culture and are neurogenic after transplantation. Stem Cells. 2006 April; 24(4):1054-64. Epub 2005 Dec 1.
da Silva Meirelles L, Chagastelles P C, Nardi N B. Mesenchymal stem cells reside in virtually all post-natal organs and tissues. J Cell Sci. 2006 Jun. 1; 119(11):2204-13.
D'Ippolito G, Diabira S, Howard G A, Menei P, Roos B A, Schiller PC. Marrow-isolated adult multilineage inducible (MIAMI) cells, a unique population of postnatal young and old human cells with extensive expansion and differentiation potential. J Cell Sci. 2004 Jun. 15; 117(Pt 14):2971-81.
Encina N R, Billotte W G, Hofmann M C. Immunomagnetic isolation of osteoprogenitors from human bone marrow stroma. Lab Invest. 1999 April; 79(4):449-57 Exp Hematol. 2002 July; 30(7):783-91.
El-Badri N S, Hakki A, Saporta S, Liang X, Madhusodanan S, Willing A E, Sanberg C D, Sanberg P R. Cord blood mesenchymal stem cells: Potential use in neurological disorders. Stem Cells Dev. 2006 August; 15(4):497-506.
Geiger M, Li R H, Friess W. Collagen sponges for bone regeneration with rhBMP-2. Adv Drug Deliv Rev. 2003 Nov. 28; 55(12):1613-29.
Giannoudis P V, Dinopoulos H, Tsiridis E. Bone substitutes: an update. Injury. 2005 November; 36 Suppl 3:S20-7
Habich A, Jurga M, Markiewicz I, Lukomska B, Bany-Laszewicz U, Domanska-Janik K. Early appearance of stem/progenitor cells with neural-like characteristics in human cord blood mononuclear fraction cultured in vitro. Exp Hematol. 2006 July; 34(7):914-25.
Huss R, Lange C, Weissinger E M, Kolb H J, Thalmeier K. Evidence of peripheral blood-derived, plastic-adherent CD34 (−/low) hematopoietic stem cell clones with mesenchymal stem cell characteristics. Stem Cells. 2000; 18(4):252-60.
Kuznetsov S A, Krebsbach P H, Satomura K, Kerr J, Riminucci M, Benayahu D, Robey P G. Single-colony derived strains of human marrow stromal fibroblasts form bone after transplantation in vivo. J Bone Miner Res 1997; 12:1335-1347
Kawamoto A, Gwon H C, Iwaguro H, Yamaguchi J I, Uchida S, Masuda H, Silver M, Ma H, Kearney M, Isner J M, Asahara T. Therapeutic potential of ex vivo expanded endothelial progenitor cells for myocardial ischemia. Circulation. 2001 Feb. 6; 103(5):634-7.
Miao D, Murant S, Scutt N, Genever P, Scutt A. Megakaryocyte-bone marrow stromal cell aggregates demonstrate increased colony formation and alkaline phosphatase expression in vitro. Tissue Eng. 2004 May-June; 10(5-6):807-17
Mitchell K E, Weiss M L, Mitchell B M, Martin P, Davis D, Morales L, Helwig B, Beerenstrauch M, Abou-Easa K, Hildreth T, Troyer D, Medicetty S. Matrix cells from Wharton's jelly form neurons and glia. Stem Cells. 2003; 21(1):50-60. Erratum in: Stem Cells. 2003; 21(2):247.
Quirici N, Soligo D, Bossolasco P, Servida F, Lumini C, Deliliers G L. Isolation of bone marrow mesenchymal stem cells by anti-nerve growth factor receptor antibodies.
Ratajczak M Z, Kucia M, Majka M, Reca R, Ratajczak J. Heterogeneous populations of bone marrow stem cells—are we spotting on the same cells from the different angles Folia Histochem Cytobiol. 2004; 42(3):139-46
Song H Y, Jeon E S, Jung J S, Kim J H. Oncostatin M induces proliferation of human adipose tissue-derived mesenchymal stem cells. Int J Biochem Cell Biol. 2005 November; 37(11):2357-65
Schueller P O, Hennersdorf M G, Strauer B E. Sudden death is associated with a widened paced QRS complex in noncoronary cardiac disease. J Interv Card Electrophysiol. 2006 March; 15(2):125-30.
Tondreau T, Lagneaux L, Dejeneffe M, Delforge A, Massy M, Mortier C, Bron D. Isolation of BM mesenchymal stem cells by plastic adhesion or negative selection: phenotype, proliferation kinetics and differentiation potential. Cytotherapy. 2004; 6(4):372-9
Verfaillie C M. Multipotent adult progenitor cells: an update. Novartis Found Symp. 2005; 265:55-61; discussion 61-5,
Vaananen H K., Mesenchymal stem cells. Ann Med. 2005; 37(7):469-79. Review.
Yanai N, Obinata M. Oncostatin m regulates mesenchymal cell differentiation and enhances hematopoietic supportive activity of bone marrow stromal cell lines. In Vitro Cell Dev Biol Anim. 2001 November-December; 37(10):698-704.
Yin T, Li L. The stem cell niches in bone. J Clin Invest. 2006 May; 116(5):1195-201
Zuk P A, Zhu M, Mizuno H, Huang J, Futrell J W, Katz A J, Benhaim P, Lorenz H P, Hedrick M H. Multilineage cells from human adipose tissue: implications for cell-based therapies. Tissue Eng. 2001 April; 7(2):211-28.

1. A method of generating and expanding tissue-progenitor cells or mature tissue cells in culture, comprising culturing intact bone marrow or intact umbilical cord tissue in a cell differentiation medium whereby tissue-progenitor cells or mature tissue cells are generated from mesenchymal stem cells and/or other progenitor cells present in the intact bone marrow or intact umbilical cord tissue and expanded.

2. The method of claim 1, wherein the cell differentiation medium is osteogenic differentiation medium and wherein the tissue-progenitor cells are bone progenitor cells.

3. The method of claim 2, wherein the osteogenic differentiation medium comprises β-Glycerophosphate, L-ascorbic acid-2-phosphate and dexamethasone.

4. The method of claim 2, wherein generation and expansion of the bone progenitor cells is confirmed by exhibition of osteoblast morphology or detection of expression of osteoblast-specific genes.

5. The method of claim 4, wherein the osteoblast-specific genes are selected from the group consisting of genes that encode RUNX 2 transcription factor, bone-specific alkaline phosphatase, procollagen aminoterminal propeptide, type I collagen, osteopontin, bone sialoprotein, osteocalcin, parathyroid hormone receptor, osteoprotegerin and receptor activator NF-KB ligand (RANKL).

6. The method of claim 2, wherein generation and expansion of the bone progenitor cells is confirmed by an increase in alkaline phosphatase (ALP) activity and calcium deposition.

7. The method of claim 2, wherein the cell differentiation medium is an osteoclast differentiation medium and wherein the tissue-progenitor cells are osteoclast progenitor cells.

8. The method of claim 1, wherein the cell differentiation medium is neurogenic differentiation medium and wherein the tissue-progenitor cells are neuronal progenitor cells.

9. The method of claim 8, wherein the neurogenic differentiation medium comprises β-mercaptoethanol, MEM non-essential amino acids, basic fibroblast growth factor (FGF), epidermal growth factor (EGF), neurotrophin-3, N2, B27 supplements, insulin, dimethylsulfoxide (DMSO), butylated hydroxyanisole (BHA), all-trans retinoic acid (RA), forskolin, valproic acid and KCl.

10. The method of claim 8, wherein generation and expansion of neuronal progenitor cells is confirmed by exhibition of neuroblast morphology or detection of neuroblast-specific genes.

11. The method of claim 8, wherein generation and expansion of neuronal progenitor cells is confirmed by detection of an increase in nestin or NCAN activity.

12. The method of claim 1, wherein the cell differentiation medium is neurogenic differentiation medium and wherein the mature tissue cells are neurons.

13. The method of claim 12, wherein the neurogenic differentiation medium comprises DMEM/F12, insulin, transferrin, selenate, NaCO₃, FGF and EGF.

14. The method of claim 12, wherein generation and expansion of neurons is confirmed by exhibition of neuron-specific morphology comprising presence of long axons and dendrites.

15. The method of claim 1, wherein the cell differentiation medium is endothelial differentiation medium and the tissue-progenitor cells are vasculature/endothelial progenitor cells.

16. The method of claim 1, wherein the cell differentiation medium is adipogenic differentiation medium and wherein the mature tissue cells are adipocytes.

17. The method of claim 1, wherein the cell differentiation medium is cardiomyogenic differentiation medium and wherein the mature tissue cells are cardiomyocytes.

18. The method of claim 1, wherein the cell differentiation medium is pancreogenic differentiation medium and wherein the tissue-progenitor cells are progenitors of pancreatic β-cells.

19. The method of claim 1, wherein the cell differentiation medium is chondrogenic differentiation medium and wherein the tissue-progenitor cells are cartilage progenitor cells.

20. The method of claim 1, wherein said culturing comprises culturing intact bone marrow.

21. The method of claim 1, wherein said culturing comprises culturing intact umbilical cord tissue.

22. The method of claim 21, wherein the umbilical cord tissue comprises Wharton's jelly.

23. The method of claim 21, wherein the umbilical cord tissue comprises umbilical cord blood.

24. The method of claim 1, wherein the cell differentiation medium comprises a cell culture medium, a corticosteroid and a reducing agent.

25. The method of claim 24, wherein the cell differentiation medium further comprises bone marrow plasma.

26. The method of claim 1, wherein conditions of said culturing comprise a temperature of 4-37° C., a humidity of atmospheric to 100% humidity; a carbon dioxide level of 0-5% CO₂ and an oxygen level of 1% oxygen to atmospheric levels.

27. The method of claim 1, wherein said culturing is conducted in the presence of a scaffold or an extracellular matrix (ECM).

28. The method of claim 27, wherein the ECM is selected from the group consisting of collagen, fibronectin, vitronectin, and laminin of a human origin.

29. The method of claim 27, wherein the ECM is derived from human peripheral blood, bone marrow or umbilical cord blood.

30. The method of claim 27, wherein the scaffold is selected from the group consisting of synthetic polymers, biological polymers of a human origin, ceramics, gels, alginates, nanofibers, mineralized and demineralized bone matrix.

31. The method of claim 1, wherein said culturing is conducted for about 2 to about 45 days.

32. The method of claim 1, wherein said culturing is conducted for about 14 days.

33. The method of claim 1, wherein the cell differentiation medium does not contain non-human based animal products.

34. The method of claim 1, wherein the intact bone marrow or the intact umbilical cord tissue and the cell differentiation medium are present in a weight ratio of from 1:1 to 1:50.

35. The method of claim 34, wherein the ratio is 1:6.

36. The method of claim 1, wherein the intact bone marrow or intact umbilical cord tissue is obtained from a human source.

37. The method of claim 34, wherein the human source is autologous.

38. The method of claim 1, wherein the intact bone marrow or the intact umbilical cord tissue is obtained from a non-human source.

39. The method of claim 1, wherein said culturing is continued until the tissue-progenitor cells or mature tissue cells become confluent.

40. A method of tissue repair or regeneration, comprising:
(a) culturing intact bone marrow or intact umbilical cord tissue in a cell differentiation medium whereby tissue-progenitor cells or mature tissue cells are generated from mesenchymal stem cells and/or other progenitor cells present in the intact bone marrow or intact umbilical cord tissue and expanded;
(b) harvesting the tissue-progenitor cells or mature tissue cells; and
(c) transplanting the tissue-progenitor cells or mature tissue cells in a patient in need thereof.

41. The method of claim 40, wherein the cell differentiation medium is osteogenic differentiation medium and wherein the tissue-progenitor cells are bone progenitor cells.

42. The method of claim 40, wherein the cell differentiation medium is neurogenic differentiation medium and wherein the tissue-progenitor cells are neuronal progenitor cells.

43. The method of claim 40, wherein the cell differentiation medium is neurogenic differentiation medium and wherein the mature tissue cells are neurons.

44. The method of claim 40, wherein the cell differentiation medium is endothelial differentiation medium and the tissue-progenitor cells are vasculature/endothelial progenitor cells.

45. The method of claim 40, wherein the cell differentiation medium is adipogenic differentiation medium and wherein the mature tissue cells are adipocytes.

46. The method of claim 40, wherein the cell differentiation medium is cardiomyogenic differentiation medium and wherein the mature tissue cells are cardiomyocytes.

47. The method of claim 40, wherein the cell differentiation medium is pancreogenic differentiation medium and wherein the tissue-progenitor cells are progenitors of pancreatic β-cells.

48. The method of claim 40, wherein the cell differentiation medium is chondrogenic differentiation medium and wherein the tissue-progenitor cells are cartilage progenitor cells.

49. The method of claim 40, wherein the cell differentiation medium does not contain non-human based animal products.

50. The method of claim 40, wherein the intact bone marrow or intact umbilical cord tissue is obtained from a human source.

51. The method of claim 50, wherein the human source is autologous.

52. A composition, comprising intact bone marrow or intact umbilical cord tissue and a cell differentiation medium, which upon culturing achieves generation and expansion of tissue-progenitor cells or mature tissue cells from mesenchymal stem cells and/or other progenitor cells present in said intact bone marrow or intact umbilical cord tissue.

53. The composition of claim 52, further comprising tissue-progenitor cells or mature tissue cells.
